(19) United States
(12) Patent Application Publication
(10) Pub. No.: US 2012/0129711 A1
(43) Pub. Date: May 24, 2012

(54) BIOMARKERS FOR THE PROGNOSIS AND HIGH-GRADE GLIOMA CLINICAL OUTCOME

(75) Inventors: JEAN MOSSER, Rennes (FR); Marie de Tayrac, Rennes (FR); Marc Aubry, Rennes (FR)

(73) Assignees: Universite de Rennes 1, Rennes (FR); Centre Hospitalier Universitaire de Rennes, Rennes (FR); Centre National de la Recherche Scientifique, Paris (FR)

(21) Appl. No.: 13/237,255

(22) Filed: Sep. 20, 2011

Related U.S. Application Data
(60) Provisional application No. 61/384,538, filed on Sep. 20, 2010.

(51) Int. Cl. C40B 30/04; G01N 33/577; C12Q 1/68

(52) U.S. Cl. 506/9; 435/6.12; 435/7.23

(57) ABSTRACT

The present invention relates to the identification and use of biomarkers with clinical relevance to high grade gliomas (HGGs). In particular, the invention provides the identity of four genes, CHAF1B, PDLIM4, EDNRB and HJURP, whose expression, at the transcriptome and proteome levels, is correlated with HGG grading and clinical survival outcome. Methods and kits are provided for using these biomarkers in the prognostication of HGGs, and in the selection and/or monitoring of treatment regimens.

### RELATED APPLICATIONS

The present application claims priority to U.S. Provisional Patent Application Ser. No. 61/384,538, which was filed on Sep. 20, 2010. The Provisional Application is incorporated herein by reference in its entirety.

### BACKGROUND OF THE INVENTION

High-grade gliomas (HGGs) are brain tumors associated with high morbidity and mortality. They are classified as either grade III or grade IV on the basis of histopathological features established by the World Health Organization (WHO) (Louis et al., Acta Neuropathol., 2007, 114(2): 97-109). In combination with other clinical parameters, the grade has long provided important prognostic information (Louis, Annu. Rev. Pathol., 2006, 1: 97-117). Recently, molecular biomarkers have been shown to be strongly associated with the prognostic of these tumors. O(6)-methylguanine-DNA-methyltransferase (MGMT) promoter hypo-methylation is involved in glioblastoma (GBM) resistance to temozolomide chemotherapy (Hegi et al., N. Engl. J. Med., 2005, 352(10): 997-1003) and mutations of the isocitrate dehydrogenase 1 (IDH1) gene are associated with better outcome of patients (Yan et al., N. Engl. J. Med., 2009, 360(8): 765-73).

Recent studies have demonstrated that molecular and genetic analysis of gliomas could help in their classification and in the design of treatment protocols (Behin et al., Lancet, 2003, 361(9354): 323-31; Li et al., Cancer Res., 2009, 69(5): 2091-9). Microarray expression profiling has characterized molecular subtypes of brain tumors associated with tumor grade, progression, and prognosis (Li et al., Cancer Res., 2009, 69(5): 2091-9; Petalidis et al., Mol. Cancer. Ther., 2008, 7(5):1013-24; Phillips et al., Cancer Cell, 2006, 9(3): 157-73; Liang et al., Proc. Natl. Acad. Sci. USA, 2005, 102(16): 5814-9; Freije et al., Cancer Res., 2004, 64(18): 6503-10, 2004; Nutt et al., Cancer Res., 2003, 63(7): 1602-7; U.S. Pat. Appln. No. 2010/0167939; and PCT Appln. No. WO 2005/042786) though only a few genes have been consistently identified (Colman et al., Arch. Neurol., 2008, 65: 877-883). To overcome such a lack of reproducibility, the best approach is to analyze multiple dataset simultaneously in order to combine the results from relevant studies. Such analysis applied to microarray data has been shown to be a powerful tool to identify candidate biomarkers and biological pathways (Hong et al., Bioinformatics, 2008, 24(3): 374-82).

The two most comprehensive glioma microarray classifications schemes published to date (Li et al., Cancer Res., 2009, 69(5): 2091-9; Liang et al., Proc. Natl. Acad. Sci. USA, 2005, 102(16): 5814-9) are based on unsupervised analysis, and they clearly show a strong association between the tumor grading and the defined glioma subtypes. These two classifications proposed by Phillips et al. (Cancer Cell, 2006, 9(3): 157-73) and Li et al. (Li et al., Cancer Res., 2009, 69(5): 2091-9) show that high-grade glioma patients with better-than-expected survival could be classified in an enriched grade III subtype designated proneural or oligodendroglioma-rich, respectively.

Therefore, there clearly still remains a need in the art for a robust signature to characterize and classify aggressive gliomas and to predict high-grade glioma clinical outcome.

### SUMMARY OF THE INVENTION

The present invention relates to improved systems and strategies for high-grade glioma classification and prognostication. In particular, the invention provides biomarkers that constitute a robust signature related to tumor aggressiveness and glioma clinical survival outcome. Indeed, the present Applicants have identified a gene prognostic classifier for high-grade gliomas (HGGs) composed of four genes: EDNRB, HJURP, CHAF1B and PDLIM4. These genes were identified, using a gene expression meta-analysis approach, as being correlated to both grading and survival of HGGs. The prognostic value of this gene classifier was validated in an independent cohort of around 200 patients by quantitative reverse transcription-PCR(RT-qPCR) and successfully compared to the prognostic power of the mutation status of the IDH1 gene and of the methylation status of the MGMT promoter. The present Applicants have also studied the expression of the EDN/RB, HJURP, p60/CAF-1 and PDLI4 proteins in HGGs, and found that the expression levels of these proteins are significantly correlated with the histological grading and with the survival outcome of HGG patients. Furthermore, they demonstrated the predictive value of integrating EDN/RB, HJURP and p60/CAF-1 immunohistological date for the prognostication of HGGs.

Accordingly, in one aspect, the present invention relates to a method for grading aggressiveness of HGG in an individual and/or providing a HGG survival outcome to an individual, the method comprising steps of: determining, in a biological sample obtained from the individual, expression levels of the four genes, CHAF1B, PDLIM4, EDNRB and HJURP, to obtain an expression pattern for the sample: and based on the expression pattern obtained, grading the aggressiveness of HGG in the individual and/or providing a HGG survival outcome for the individual.

In certain embodiments, the individual tested is receiving or has received a treatment for HGG and the method is used for monitoring or assessing the treatment's effects on HGG aggressiveness and/or HGG survival outcome in the individual treated.

In certain embodiments, determining the expression levels of the four genes in a method of the invention comprises determining mRNA expression level for each of said four genes; and normalizing the mRNA expression levels determined in relation to the mRNA expression levels of one or more reference genes. The reference genes may be house keeping genes, such as HPRT1 (hypoxanthine phosphoribosyltransferase) and B2M (beta-2 microglobulin).

In a method of the invention, determining the expression levels of the four genes may comprise performing a quantitative polymerase chain reaction or a microarray analysis or a next-generation sequencing method. In certain embodiments, determining the expression levels of the four genes further comprises calculating a gene expression risk score according to a Cox proportional hazard risk equation.

Overexpression of EDNRB correlates with less aggressive HGG and longer survival outcome, and overexpression of CHAF1B, PDLIM4, and HJURP correlates with more aggressive HGG and shorter survival outcome.

In certain embodiments, a method of the invention further comprises a step of determining, in the biological sample, the methylation status of the MGMT promoter and/or the mutation status of IDH1.

In other embodiments, determining the expression levels of the four genes in a method of the invention comprises determining the expression levels of the four proteins, p60/CAF-1, PDLI4, EDN/RB and HJURP, encoded by the four genes. The expression levels of the proteins may be determined by performing an immunoassay. Overexpression of the four proteins correlates with more aggressive HGG and shorter survival outcome.

In a method according to the invention, the biological sample may be any suitable biological sample, such as, for example, a fixed, paraffin-embedded tissue sample, a fresh tissue sample, or a frozen tissue sample.

In another aspect, the present invention provides a method for grading aggressiveness of HGG in an individual an/or providing a HGG survival outcome to an individual, the method comprising steps of: determining, in a biological sample obtained from the individual, expression levels of at least one protein selected from the group consisting of p60/CAF-1, PDLI4, EDN/RB and HJURP, or of the three proteins, p60/CAF-1, EDN/RB and HJURP, to obtain a protein expression pattern for the sample; and based on the protein expression pattern obtained, grading the aggressiveness of HGG in the individual and/or providing a HGG survival outcome for the individual.

In certain embodiments, the individual tested is receiving or has received a treatment for HGG and the method is used for monitoring or assessing the treatment's effects on HGG aggressiveness and/or HGG survival outcome in the individual treated.

In a method of the invention, determining the protein expression level may comprise performing an immunoassay. Overexpression of any one of the four proteins correlates with more aggressive HGG and shorter survival outcome. Overexpression of the three proteins, p60/CAF-1, EDN/RB and HJURP, correlates with more aggressive HGG and shorter survival outcome.

In a method according to the invention, the biological sample may be any suitable biological sample, such as, for example, a fixed, paraffin-embedded tissue sample, a fresh tissue sample, or a frozen tissue sample.

In yet another aspect, the present invention provides a kit for grading aggressiveness of HGG and/or providing a HGG survival outcome to an individual, said kit comprising: reagents that specifically detect expression levels of the four genes, CHAF1B, PDLIM4, EDNRB and HJURP, or at least on reagent that specifically detects the expression level of at least one of the four proteins, p60/CAF-1, PDLI4, EDN/RB and HJURP, or reagents that specifically detect expression levels of the three proteins, p60/CAF-1, EDN/RB and HJURP.

In certain embodiments, a kit further comprises instructions for grading aggressiveness of HGG and/or providing a HGG survival outcome to an individual according to a method of the invention.

In certain embodiments, reagents that specifically detect expression levels of the four genes, CHAF1B, PDLIM4, EDNRB and HJURP, are nucleic acid probes complementary to mRNA of said genes. These nucleic acid probes may or may not be immobilized on a substrate surface.

In certain embodiments, the at least one reagent that specifically detects the expression level of at least one of the four proteins: p60/CAF-1, PDLI4, EDN/RB and HJURP, and the reagents that specifically detect expression levels of the three proteins: p60/CAF-1, EDN/RB and HJURP, are antibodies that specifically bind to one of the proteins.

These and other objects, advantages and features of the present invention will become apparent to those of ordinary skill in the art having read the following detailed description of the preferred embodiments.

### BRIEF DESCRIPTION OF THE DRAWING

FIG. 1 is a scheme of the analysis workflow. [symbol] Meta-analysis was performed on three publicly available HGGs microarray datasets (267 patients) to define a robust signature related to tumor aggressiveness (grade III versus grade IV). [symbol] This signature was used to define genes also associated with outcome by survival analysis. This was performed on 144 of the 267 patients for which survival data was available. [symbol] Genes associated with both grading and outcome were used to select an optimal survival model. This model was based on the weighted expression of four genes (risk-score). [symbol] Two independent validations were performed: the first, on a publicly available microarray study, and the second, on the local cohort of HGGs, by quantitative reverse transcription-PCR(RT, qPCR). [symbol] Model performances were assessed on the patients from the local cohort with full clinical and biological data (176 of 194 patients).

FIG. 2 is a set of graphs showing Kaplan-Meier estimates of Overall Survival after subdivision into low and high risk-score groups. (A) Training cohort of 144 patients with malignant glioma, analyzed by microarray meta-analysis (GEO Datasets: GSE4271 and GSE4412). (B) Validation cohort of 56 patients with malignant gliomas reported by Petalidis et al. (2008). (C) Whole anaplastic astrocytoma set (n=46). (D) Whole glioblastoma set (n=154).

FIG. 3 is a set of graphs showing the survival of patients with High-Grade Glioma according to the four-gene risk-score, the MGMT promoter methylation status and the IDH1 mutational status. (A) Kaplan-Meier estimates of overall survival in the whole local cohort after subdivision into two groups (low and high risk of death) on the basis of the risk-score model, with log2-transformed data issued from quantitative reverse-transcriptase polymerase chain reaction analysis. The overall survival among low-risk patients is 55.8 months (95% CI, 26.0 to not reached), as compared with 14.5 months (95% CI, 12.5 to 16.0) among high-risk patients (P<0.001). (B) Kaplan-Meier estimates of overall survival in the whole local cohort after subdivision into two groups depending on the DNA methylation status of the MGMT promoter. Median survival is 19.5 months (95% CI, 16.7 to 29.4) for patients with tumoral methylated MGMT promoter and 14.5 months (95% CI, 11.4 to 16.2) for patients with tumoral unmethylated MGMT promoter. (C) Kaplan-Meier estimates of overall survival in the whole local cohort after subdivision into two groups depending on the presence of IDH1 mutations. IDH1 mutational status is significantly associated with the overall survival in all cohorts (P<0.001, median survival not reached [95% CI, 42.5 to not reached] versus 14.9 months [95% CI, 13.7 to 16.5]).

FIG. 4 is a graph showing the combined stratification based on the IDH1 mutational status and the four-gene risk-score. Three groups of HGGs (good-, intermediate- and poor-outcome groups) with significant differences in OS (P<0.001) are defined by the combination of the IDH1 mutational status and the four-gene risk-score. The group of HGGs with intermediate-outcome (non-mutated/low-risk or mutated/high-risk) is characterized by a median survival of 20.6 months (95% CI, 16.5 to 72.1), as compared to 14 months (95% CI, 12.3 to 15.2) for the poor-outcome group (non mutated/high-risk) and to a median survival not reached (95% CI, 83.2 to not reached) for the good-outcome group (mutated/low-risk).

FIG. 5 is a set of pictures showing examples of the range of markers immunopositivity within normal adult brain and high-grade gliomas. Sections of paraffin-embedded specimens of a total of 6 normal brain tissues and 96 HGGs specimens including WHO grade III to IV glioma samples were stained by immunohistochemistry using an anti-EDN/RB, HJURP, p60/CAF-1 and PDLI4 antibodies. Representative data are reported for each staining: a section of normal adult brain tissue, a section of tumor with low-level positivity and a section of tumor with high-level positivity.

FIG. 6 is a graph showing the results of immunohistochemical analyses of markers expression in grade III and grade IV gliomas. Statistical quantification of the average mean absorbance of each marker staining between grade III (32 cases) and grade IV specimens (64 cases) are presented. P-values were obtained by applying a Student t-test for each comparison.

FIG. 7 is a set of graphs showing the results of overall survival analyses of molecular markers. Kaplan-Meier estimates of overall survival are presented for all markers (EDN/RB, HJURP, p60/CAF-1 and PDLI4) after subdivision of the cohort of patients into two groups (low and high risk of death) on the basis of the cut-offs defined by analyses of the time-dependent ROC curves. (A) For the EDN/RB protein, the overall survival among low-risk patients is 18.5 months (95% CI, 14.9-69.7), as compared with 14 months (95% CI, 10.4-18.3) among high-risk patients (P=0.007). (B) For the HJURP protein, the difference in overall survival between low-risk and high-risk patients is significant (P=0.01 with 38.8 months [95% CI, 29.4-12.5] versus 14.9 months [95% CI, 12.5 to 17], respectively). (C) For the p60/CAF-1 protein, the difference in overall survival between high expression level patients and low expression level patients was also significant (p=0.004, 14 months [95% CI, 11.4-16.2] versus 23.5 months [95% CI, 16.8-55.8], respectively). (D) For the PDLI4 protein, the difference was also significant (P=0.02, 14.9 months [95% CI, 13-18.2] versus 19.6 months [95% CI, 16.7-Inf]).

FIG. 8 presents a summary of EDNRB, HJURP, p60/CAF-1 and PDLI4 immunohistochemistry results obtained.

### DEFINITIONS

Throughout the specification, several terms are employed that are defined in the following paragraphs.

The terms “subject” and “individual” are used herein interchangeably. They refer to a human being who may or may not suffer from high-grade glioma (HGG). In many embodiments of the present invention, the subject has been diagnosed with HGG. In such embodiments, the subject may also be called “patient”. The terms “subject”, “individual” and “patient” do not denote a particular age.

The terms “biomarker” and “marker” are used herein interchangeably. They refer to a substance that is a distinctive indicator of a biological process, biological event and/or pathological condition. As used herein, the term “biomarker of HGG” refers to a gene or a protein according to the present invention whose expression is indicative of HGG aggressiveness and/or progression (and therefore HGG grading), and predictive of survival outcome.

The term “biological sample” is used herein in its broadest sense. A biological sample is generally obtained from a subject. A sample may be of any biological tissue or fluid with which biomarkers of the present invention may be assayed. Frequently, a sample will be a “clinical sample”, i.e., a sample derived from a patient. Examples of biological samples suitable for use in the present invention include, but are not limited to, bodily fluids, e.g., blood samples (e.g., blood smears) and cerebrospinal fluid; brain tissue samples or bone marrow tissue samples such as tissue or fine needle biopsy samples, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample, as well as proteins or nucleic acid molecules extracted from the sample. Processing of a biological sample may involve one or more of: filtration, distillation, extraction, concentration, inactivation of interfering components, addition of reagents, and the like.

As used herein, the term “gene” refers to a polynucleotide that encodes a discrete macromolecular product, be it a RNA or a protein, and may include regulatory sequences preceding (5′ non-coding sequences) and following (3′ non-coding sequences) the coding sequence. As more than one polynucleotide may encode a discrete product, the term also include alleles and polymorphisms of a gene that encode the same product, or a functionally associated (including gain, loss, or modulation of function) analog thereof.

The term “gene expression” refers to the process by which RNA and proteins are made from the instructions encoded in genes. Gene expression includes transcription and/or translation of nucleic acid material. The terms “gene expression pattern” and “gene expression profile” are used herein interchangeably. They refer to the expression of an individual gene or of a set of genes. A gene expression pattern may include information regarding the presence of target transcripts in a sample, and the relative or absolute abundance levels of target transcripts.

The term “differentially expressed gene”, as used herein, refers to a gene whose level of expression is different at different grades of high-grade glioma (e.g., grade III vs. grade IV) and/or different for different survival outcomes of high-grade glioma patients. As will be appreciated by those skilled in the art, a gene may be differentially expressed at the nucleic acid level and/or at the protein level, or may undergo alternative splicing resulting in a different polypeptide product. Differential expression includes quantitative, as well as qualitative, differences in the temporal or cellular expression pattern in a gene or its expression products. As described in greater details below, a differentially expressed gene, alone or in combination with other differentially expressed genes, is useful in a variety of different applications in diagnostic, therapeutic, prognosis, drug development and related areas. The expression patterns of the differentially expressed genes disclosed herein can be described as a fingerprint or a signature of HGG progression. They can be used as a point of reference to compare and characterize unknown biological samples and biological samples for which further information is sought.

The term “RNA transcript” refers to the product resulting from transcription of a DNA sequence. When the transcript is the original, unmodified product of a RNA polymerase catalyzed transcription, it is referred to as the primary transcript. An RNA transcript that has been processed (e.g., spliced, etc) will differ in sequence from the primary transcript. A processed RNA transcript that is translated into protein is often called a messenger RNA (mRNA). The term “messenger RNA or mRNA” refers to a form of RNA that serves as a template to direct protein biosynthesis. Typically, the amount of any particular type of mRNA (i.e., having the same sequence, and originating from the same gene) represents the extent to which a gene has been expressed.

The term “complementary DNA or cDNA” refers to a DNA molecule that is complementary to mRNA. cDNAs can be made by DNA polymerase (e.g., reverse transcriptase) or by direct chemical synthesis. The term “complementary” refers to nucleic acid sequences that base-pair according to the standard Watson-Crick complementary rules, or that are capable of hybridizing to a particular nucleic acid segment under relatively stringent conditions. Nucleic acid polymers are optionally complementary across only portions of their entire sequences.

The term “hybridizing” refers to the binding of two single stranded nucleic acids via complementary base pairing. The terms “specific hybridizing” and “specific binding” are used herein interchangeably. They refer to a process in which a nucleic acid molecule preferentially binds, duplexes or hybridizes to a particular nucleic acid sequence under stringent conditions (e.g., in the presence of competitor nucleic acids with a lower degree of complementarity to the hybridizing strand). In certain embodiments of the present invention, these terms more specifically refer to a process in which a nucleic acid fragment (or segment) from a test sample preferentially binds to a particular genetic probe and to a lesser extent or not at all, to other genetic probes, for example, when these genetic probes are immobilized on an array.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is a full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains such as oxidation of sulfhydryl groups. Thus, the term “protein” (or its equivalent terms) is intended to include the amino acid sequence of the full-length native protein or a fragment thereof, subject to those modifications that do not significantly change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

The term “protein fragment”, as used herein, refers to a polypeptide comprising an amino acid sequence of at least 5 consecutive amino acid residues (preferably at least about: 10, 15, 20, 25, 30, 40, 50, 60, 70, 80, 90, 100, 125, 150, 175, 200, or 250 consecutive amino acid residues) of the amino acid sequence of the protein. The fragment of a protein may or may not possess a functional activity of the protein.

The terms “array”, “micro-array”, and “biochip” are used herein interchangeably. They refer to an arrangement, on a substrate surface, of hybridizable array elements, preferably, multiple nucleic acid molecules of known sequences. Each nucleic acid molecule is immobilized to a discrete spot (i.e., a defined location or assigned position) on the substrate surface. The term “micro-array” more specifically refers to an array that is miniaturized so as to require microscopic examination for visual evaluation. The term “gene expression array” refers to an array comprising a plurality of genetic probes immobilized on a substrate surface that can be used for quantitation of mRNA expression levels. The term “genetic probe”, as used herein, refers to a nucleic acid molecule of known sequence, which has its origin, in a defined region of the genome and can be short DNA sequence (i.e., an oligonucletide), a PCR product, or mRNA isolate. Genetic probes are genetic-specific DNA sequences to which nucleic acid fragments from a test sample are hybridized. Genetic probes specifically bind to nucleic acids of complementary or substantially complementary sequence through one or more types of chemical bonds, usually through hydrogen bond formation.

As used herein, the term “a reagent that specifically detects expression levels” refers to one or more reagents used to detect the expression of one or more genes. Examples of suitable reagents include, but are not limited to, nucleic acid probes capable of specifically hybridizing to the gene of interest or mRNA transcripts thereof, PCR primers capable of specifically amplifying the gene of interest or mRNA transcripts thereof, and antibodies capable of specifically binding to proteins encoded by the gene of interest. The term “amplify” is used in the broad sense to mean generating an amplification product. “Amplification”, as used herein, generally refers to the process of producing multiple copies of a desired sequence, particularly those of a sample. A “copy” does not necessarily mean perfect sequence complementarity or identity to the template sequence.

The term “treatment” is used herein to characterize a method that is aimed at (1) delaying or preventing the onset of a disease or condition (here high-grade glioma); or (2) slowing down or stopping the progression, aggravation, or deteriorations of the symptoms of the condition; or (3) bringing about ameliorations or the symptoms of the condition; or (4) curing the condition. A treatment for high-grade glioma is generally administered after initiation of the disease, for a therapeutic action.

The terms “approximately” and “about”, as used in reference to a number, generally include numbers that fall within a range of 10% in either direction of the number (greater than or less than the number) unless otherwise stated or otherwise evident from the context (except where such number would exceed 100% of a possible value).

### DETAILED DESCRIPTION OF CERTAIN PREFERRED EMBODIMENTS

As mentioned above, the present invention provides biomarkers whose expression at the transcriptome and proteome levels correlate with the grading of high grade gliomas and with the survival outcome of glioma patients. Also provided are methods, arrays and kits for using these biomarkers for the prognosis of high-grade glioma progression in patients.

### I—Biomarkers

In one aspect, the present invention provides the identity of a set of four genes (EDNRB, HJURP, CHAF1B and PDLIM4) and of the four proteins (EDB/RB, HJURP, p60/CAF-1 and PDLI4) encoded by these genes, whose expression pattern is indicative of HGG grading and survival outcome in HGG patients.

As used herein, the term “EDNRB” refers to the human gene that encodes the endothelin receptor type B. The EDNRB gene is located on the long (q) arm of chromosome 13 at position 22 (GenBank RefSeqGene: NG_{—}011630.2; RefSeq (mRNA): NM_{—}001201397.1, NM_{—}001122659.2, NM_{—}003991.3, NM_{—}000115.3). EDNRB is also known as ETB, ET-B, ETBR, ETRB, HSCR, WZ4A, ABCDS, or HSCR2. As used herein, the term “EDN/RB” refers to the endothelin receptor type B (UniProt: P24530) encoded by the human EDNRB gene. EDN/RB is a G protein-coupled receptor which activates a phosphatidylinositol-calcium second messenger system. Its ligand, endothelin, consists of three potent vasoactive peptides (ET1, ET2 and ET3). EDN/RB is known to be implicated in cell proliferation, survival, invasion, angiogenesis and metastasis. The Applicants have found that over-expression of EDNRB correlates with better prognosis in terms of survival outcome in HGG patients. On the other hand, over-expression of EDN/RB was found to be associated with a pejorative evaluation of HGGs.

As used herein, the term “HJURP” refers to the human gene that encodes the Holliday junction recognition protein. The HJURP gene is located on the long (q) arm of chromosome 2 at position 37 (GenBank RefSeqGene: NG_{—}000002.11; RefSeq (mRNA): NM_{—}018410.3). HJURP is also known as FAKTS, URLC9, hFLEG1, or DKFZp762E1312. As used herein, the term “HJURP” refers to the Holliday junction recognition protein (UniProt: □8NCD3) encoded by the human HJURP gene. HJURP is a centrometric protein that has been shown to be an indispensable factor for cell-cycle regulation of centromeric chromatic assembly and for chromosomal stability in immortalized cancer cells. The Applicants have found that over-expression of HJURP, at the genome, transcriptome and proteome levels, is associated with higher grade of HGGs and shorter survival outcome in HGG patients.

As used herein, the term “CHAF1B” refers to the human gene that encodes the chromatin assembly factor 1, subunit B (p60) (p60/CAF-1). The CHAF1B gene is located on the long (q) arm of chromosome 21 at position 22 (GenBank RefSeqGene: NC_{—}000021.8; RefSeq (mRNA): NM_{—}005441.2). CHAF1B is also known as CAF1, MPP7, CAF-1, CAF1A, CAF1P60, CAF-IP60, or MPHOSPH7. As used herein, the term “p60/CAF-1” refers to the chromatin assembly factor 1, subunit B protein (UniProt: Q13112) encoded by the human CHAF1B gene. The p60/CAF-1 protein is one of the three subunits forming the chromatin assembly factor I (CAF-1) with p48 and p150. CAF-1 plays a major role in chromatin assembly after replication and DNA repair. The Applicants have found that over-expression of CHAF1B, at the transcriptome and proteome levels, is associated with higher grade of HGGs and shorter survival outcome in HGG patients.

As used herein, the term “PDLIM4” refers to the human gene that encodes the PDZ and LIM domain protein 4 (PDLI4). The PDLIM4 gene is located on the long (q) arm of chromosome 5 at position 31 (GenBank RefSeqGene: NC_{—}015836.1 RefSeq (mRNA): NM_{—}001131027.1, NM_{—}003687.3). PDLIM4 is also known as RIL. As used herein the term “PDLI4” refers to the PDZ and LIM domain protein (UnitProt: P50479) encoded by the human PDLIM4 gene. PDLI4 is a regulator of actin stress fiber turnover. The Applicants have found that over-expression of PDLIM4, at the transcriptome and proteome levels, is associated with higher grade of HGGs and shorter survival outcome in HGG patients.

### II—Prognosis Methods

As will be appreciated by those of ordinary skill in the art, biomarkers whose expression profiles correlate with HGG grading and survival outcome can be used to characterize biological samples of patients and thereby provide prognosis. Accordingly, the present invention provides methods for characterizing biological samples obtained from patients diagnosed with HGG, for assessing advancement and/or aggressiveness of HGG in patients and/or for predicting clinical survival outcome of patients affected by HGG.

The terms “high-grade glioma” and “HGG” are used herein interchangeably. They refer to gliomas that are grade III or grade IV according to the WHO grading system. Such clinical conditions include glioblastoma multiforme, anaplastic astrocytoma, anaplastic oligoastrocytoma, and higher grade oligodendrogliomas.

### Biological Samples

The methods described herein may be applied to the study of any biological sample allowing biomarkers of the invention to be assays. Examples of such biological samples include in particular samples of brain tissue, bone marrow tissue, cerebrospinal fluid or blood, as wells as cells (or their progeny) or cell content isolated from such tissues or fluids. Tissue samples may be fresh or frozen samples, or paraffin-embedded samples collected from a subject, or archival tissue samples, for example, with known diagnosis, treatment and/or outcome history. Biological samples may be collected by any non-invasive means, such as, for example, fine needle aspiration and needle biopsy, or alternatively, by an invasive method, including for example, surgical biopsy.

In certain embodiments, the inventive methods are performed on the biological sample itself without processing of the sample or with limited processing of the sample, e.g., after embedding the sample in paraffin after fixing with a fixing agent such as formalin.

In other embodiments, the inventive methods are performed at the cell level (e.g., after isolation of cells from the biological sample). However, in such embodiments, the inventive methods are preferably performed using a sample comprising many cells, where the assay is “averaging” expression over the entire collection of cells present in the sample. Preferably, there is enough of the brain or bone marrow tissue sample to accurately and reliably determine the expression levels of the set of biomarkers of interest. Multiple biological samples may be taken from the same tissue/body part in order to obtain a representative sampling of the tissue.

In still other embodiments, the inventive methods are performed on nucleic acid or protein extracts prepared from the biological sample.

For example, RNA may be extracted from the brain or bone marrow tissue sample and analyzed using a method of the invention. Methods of RNA extraction are well known in the art (see, for example, J. Sambrook et al., “Molecular Cloning: A Laboratory Manual”, 1989, 2nd Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidium thiocyanate and/or beta-mercaptoethanol, which are known to act as RNase inhibitors. Isolated total RNA may then be further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations.

Numerous different and versatile kits can be used to extract RNA (i.e., total RNA or mRNA) from human bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Giagen, Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase).

In certain embodiments, the RNA isolated from the biological sample (for example, after amplification and/or conversion to cDNA or cRNA) is labeled with a detectable agent before being analyzed. The role of a detectable agent is to facilitate detection of RNA or to allow visualization of hybridized nucleic acid fragments (e.g., nucleic acid fragments hybridized to genetic probes in an array-based assay). Preferably, the detectable agent is selected such that it generates a signal which can be measured and whose intensity is related to the amount of labeled nucleic acids present in the sample being analyzed. In array-based analysis methods, the detectable agent is also preferably selected such that is generates a localized signal, thereby allowing spatial resolution of the signal from each spot on the array.

Methods for labeling nucleic acid molecules are well known in the art. For a review for labeling protocols, label detection methods and developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those use in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

The inventive methods may also be performed on a protein extract from the biological sample. Preferably, the protein extract contains the total protein content. However, the methods may also be performed on extracts containing one or more of: membrane proteins, nuclear proteins, and cytosolic proteins. Methods of protein extraction are well known in the art (see, for example “Protein Methods”, D. M. Bollag et al., 2nd Ed., 1996, Wiley-Liss; “Protein Purification Methods: A Practical Approach”, E. L. Harris and S. Angal (Eds.), 1989; “Protein Purification Techniques: A Practical Approach”, S. Roe, 2nd Ed., 2001, Oxford University Press; “Principles and Reactions of Protein Extraction, Purification, and Characterization”, H. Ahmed, 2005, CRC Press: Boca Raton, Fla.). Different kits can be used to extract proteins from bodily fluids and tissues that are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kit(s) most appropriate for a particular situation. After the protein extract has been obtained, the protein concentration of the extract is preferably standardized to a value being the same as that of the control sample in order to allow signals of the protein markers to be quantified. Such standardization can be performed using photometric or spectrometric methods or gel electrophoresis.

### Determination of Protein Expression Levels

The prognosis methods of the invention generally involve determination, in a biological sample obtained from a HGG patient, of the expression levels of the inventive biomarkers. In certain embodiments, the expression levels of the four proteins EDN/RB, HJURP, p60/CAF-1 and PDLI4 are determined. In other embodiments, the expression levels of the three proteins p60/CAF-1, EDN/RB and HJURP are determined. In yet other embodiments, the expression level of at least one of the proteins EDN/RB, HJURP, p60/CAF-1 and PDLI4 is determined.

Determination of protein expression levels in the practice of the inventive methods may be performed by any suitable method (see, for example, E. Harlow and A. Lane, “Antibodies: A Laboratories Manual”, 1988, Cold Spring Harbor Laboratory: Cold Spring Harbor, N.Y.).

Binding Agents. In general, protein expression levels of are determined by contacting a biological sample isolated from a patient with binding agents for one or more of the protein biomarkers; detecting, in the sample, the levels of proteins that bind to the binding agents; and comparing the levels of proteins in the sample with the levels of the proteins in a control sample or with the levels of referenced proteins. As used herein, the term “binding agent” refers to an entity such as a polypeptide or antibody that specifically binds to an inventive protein biomarker. An entity “specifically binds” to a protein if it reacts/interacts at a detectable level with the protein but does not react/interact with polypeptides containing unrelated sequences or sequences of different polypeptides.

In certain embodiments, the binding agent is a ribosome, with or without a peptide component, an RNA molecule, or a polypeptide (e.g., a polypeptide that comprises an amino acid sequence of a protein biomarker, a variant thereof, or a non-peptide mimetic of such sequence).

In other embodiments, the binding agent is an antibody specific for a protein marker of the invention. Suitable antibodies for use in methods of the invention include monoclonal and polyclonal antibodies, immunologically active fragments (e.g., Fab or (Fab)2 fragments), antibody heavy chains, humamized antibodies, antibody light chains, and chimeric antibodies. Antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czernik et al., Neuroprotocols, 1995, 6: 56-61; H. Zhang et al., J. Biol. Chem. 2002, 277: 39379-39387; S. L. Morrison et al., Proc. Natl. Acad. Sci., 1984, 81: 6851-6855; M. S. Neuberger et al., Nature, 1984, 312: 604-608; S. Takeda et al., Nature, 1985, 314: 452-454). Antibodies to be used in the methods of the invention can be purified by methods well known in the art (see, for example, S. A. Minden, “Monoclonal Antibody Purification”, 1996, IBC Biomedical Library Series: Southbridge, Mass.). For example, antibodies can be affinity-purified by passage over a column to which a protein biomarker of the invention, or fragment thereof, is bound. The bound antibodies can then be eluted from the column using a buffer with a high salt concentration.

Instead of being prepared, antibodies to be used in the methods of the present invention may be obtained from scientific or commercial sources. Examples of commercially available anti-EDN/RB antibodies include, but are not limited to, the rabbit or sheep anti-human EDN/RB antibodies from LifeSpan Biosciences, and the mouse anti-human EDN/RB antibodies from Immuno-Biological Laboratories. Examples of commercially available anti-HJURP antibodies include, but are not limited to, the rabbit anti-human HJURP antibodies from SigmaAldrich or from Atlas Antibodies. Examples of commercially anti-p60/CAF-1 antibodies include, but are not limited to, the mouse anti-human p60/CAF-1 antibodies from SigmaAldrich or Abcam or Thermo Scientific Pierce Antibodies or EMD Millipore or Novus Biologicals, and the rabbit anti-human p60/CAF-1 antibodies from Abcam or Bethyl Laboratories. Examples of commercially anti-PDLI4 antibodies include, but are not limited to, the mouse anti-human PDLI4 antibodies from SigmaAldrich and the goat anti-human PDLI4 antibodies from LifeSpan Biosciences.

Labeled Binding Agents. Preferably, the binding agent (e.g., antibody) is directly or indirectly labeled with a detectable moiety. The role of a detectable agent is to facilitate the detection step of the prognosis method by allowing visualization of the complex formed by reaction or association between the binding agent and the protein biomarker (or analog or fragment thereof). Preferably, the detectable agent is selected such that is generates a signal which can be measured and whose intensity is related (preferably proportional) to the amount of protein biomarker present in the sample being analyzed. Methods for labeling biological molecules such as polypeptides and antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors, and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens, and proteins for which antisera or monoclonal antibodies are available.

In certain embodiments, the binding agents (e.g., antibodies) may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex, Sepharose, liposomes, carboxymethyl cellulose, polyacrylamydes, polystyrene, gabbros, filter paper, magnetite, ion-exchange resin, plastic film, plastic tube, glass, polyamine-methyl vinyl-ether-maleic acid copolymer, amino acid copolymer ethylene-maleic acid copolymer, nylon, silk, and the like. Binding agents may be indirectly immobilized using second binding agents specific for the first binding agents (e.g., a mouse antibody specific for a protein biomarker may be immobilized using an sheep anti-mouse IgG Fc fragment specific antibody coated on the carrier or support).

Protein expression levels in the prognosis methods of the present invention may be determined using immunoassays. Examples of such assays are radioimmunoassay, enzyme immunoassays (e.g., ELISA), immunofluorescence, immunoprecipitation, latex agglutination, hemagglutination, and histochemical tests, which are conventional methods well-known in the art. As will be appreciated by one skilled in the art, the immunoassay may be competitive or non-competitive. Methods of detection and quantification of the signal generated by the complex formed by reaction or association of the binding agent with the protein biomarker will depend on the nature of the assay and of the detectable moiety (e.g., fluorescent moiety).

Alternatively, the protein expression levels may be determined using mass spectrometry-based methods of image-based (including use of labeled ligand) methods known in the art for the detection of proteins. Other suitable methods include proteomics-based methods. Proteomics, which studies the global changes of protein expression in a sample, typically includes the following steps: (1) separation of individual proteins in a sample, for example by electrophoresis (1D-PAGE), (2) identification of individual proteins recovered, for example by mass spectrometry or N-terminal sequencing, and (3) analysis of the date using bioinformatics.

### Determination of Polynucleotide Expression Levels

In other embodiments, determination, in a biological sample obtained from a HGG patient, of the expression levels of the inventive biomarkers is performed by determining the expression levels of the four genes: EDNRB, HJURP, CHAF1B and PDLIM4.

Determination of expression levels of nucleic acid molecules in the practice of the inventive methods may be performed by any suitable method, including, but not limited to, Southern analysis, Northern analysis, polymerase chain reaction (PCR) (see, for example, U.S. Pat Nos. 4,683,195; 4,683,202, and 6,040,166; “PCR Protocols: A Guide to Methods and Applications”, Innis et al. (Eds.), 1990, Academic Press: New York), reverse transcriptase PCR (RT-PCT) in particular quantitative reverse transcriptase PCR, anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan™, and the like. Other suitable methods include the next generation sequencing technologies which allow for deep sequencing, such as for example RNA-seq (also called Whole Transcriptome Shotgun Sequencing or WTSS).

Nucleic acid probes for use in the detection of polynucleotide sequences in biological samples may be constructed using convention methods known in the art. Suitable probes may be based on nucleic acid sequences from a gene biomarker, preferably comprising between 15 to 40 nucleotides. A nucleic acid probe may be labeled with a detectable moiety, as mentioned above. The association between the nucleic acid probe and detectable moiety can be covalent or non-covalent. Detectable moieties can be attached directly to the nucleic acid probes or indirectly through a linker (E. S. Mansfield et al., Mol. Cell. Probes, 1995, 9: 145-156). Methods for labeling nucleic acid molecules are well-known in the art (for a review of labeling protocols, and label detection techniques, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153).

Nucleic acid probes may be used in hybridization techniques to detect the gene biomarkers or their RNA products. The technique generally involves contacting and incubating nucleic acid molecules isolated from a biological sample obtained from a HGG patient with the nucleic acid probes under conditions such that specific hybridization can take place between the nucleic acid probes and the complementary sequences of the nucleic acid molecules. After incubation, the non-hybridized nucleic acid molecules are removed, and the presence and amount of nucleic acids that have hybridized to the probes are detected and quantified.

Detection of nucleic acid molecules may involve amplification of specific polynucleotide sequences using an amplification method such as PCR, followed by analysis of the amplified products using techniques known in the art. Suitable primers can be routinely designed by one skilled in the art. In order to maximize hybridization under assay conditions, primers and probes employed in the methods of the invention generally have at least 60%, preferably at least 75% and more preferably at least 90% identity to a portion of the gene biomarker.

Hybridization, amplification, and/or next generation sequencing techniques described herein may be used to determine the expression levels of the gene biomarkers of the invention.

Alternatively, obligonucleotides or longer fragments derived from the genes may be used as probes in a microarray. A number of different array configuration and methods for their preparation are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

In certain embodiments, a method of the present invention further comprises determining at least one of: the methylation status of the MGMT promoter and the mutational status of IDH1. Methods for determining the methylation status of the MGMT promoter (Hegi et al., N. Engl. J. Med., 2005, 352(10): 997-1003) and for determining the mutational status of IDH1 (Yan et al., N. Engl. J. Med., 2009, 360(8): 765-73) are known in the art.

### HGG Aggressiveness Grading and Survival Outcome Prognosis

Once the expression levels of the biomarkers have been determined (for example as described above) for the biological sample being tested, they may be compared to the expression levels in one or more control or reference samples or to at least expression profile map for HGG.

As known in the art, comparison of expression levels according to methods of the present invention is preferably performed after the expression levels obtained have been corrected for both differences in the amount of sample assayed and variability in the quality of the sample used (e.g., amount of protein extracted or number of cells stained, or amount and quality of mRNA tested). Correction may be carried out using any suitable method well-known in the art. For example, the protein concentration of a sample may be standardized using photometric or spectrometric methods or gel electrophoresis (as already mentioned above) or via cell counting before the sample is analyzed. For analyses performed on nucleic acid molecules, correction may be carried out by normalizing the levels against reference genes (e.g., housekeeping genes such as, for example, the B2M (β-2-microglobulin) gene and the HPRT¹ (hypoxanthine phosphoribosyltransferase) gene) in the same sample. Alternatively or additionally, normalization can be based on the mean or median signal (e.g., Ct in the case of RT-PCR) of all assayed genes (global normalization approach).

Normalized expression levels of the biomarkers or biomarker combinations determined for a biological sample to be tested according to a method of the invention may be compared to the normalized expression levels of the same biomarkers or biomarker combinations determined in one or more control or reference biological samples. Reference samples may be obtained from healthy individuals and from individuals afflicted with HGG (in particular HGG patients with known grading and survival outcome, for example a HGG patient cohort). Reference expression levels of biomarkers or biomarker combinations of the invention are preferably determined for a significant number of HGG patients, and an average is obtained. Reference expression levels of biomarkers or biomarker combinations obtained from a large number of HGG patients may be computed in a HGG grading and/or HGG survival outcome expression profile map.

An HGG aggressiveness grading and/or HGG survival outcome expression profile map is a representation of the expression levels of biomarkers or biomarker combinations of the present invention that are predictive of the aggressiveness of HGG and/or that are predictive of survival outcome (e.g., period of time in months or years) of a patient affected with HGG. The map may be presented as a graphical representation (e.g., on a paper or a computer screen), a physical representation (e.g., a gel or array) or a digital representation stored in a computer-readable medium. Each map may correspond to a particular HGG aggressiveness and/or survival outcome. Alternatively, an HGG expression profile map may define delineations made based upon all the HGG expression profile maps obtained on a cohort. The results obtained from a HGG patient cohort may be summarized in a HGG grading or HGG survival outcome expression profile map containing gene expression risk scores calculated according to a Cox risk equation. The hazard function for the Cox proportional hazard model has the following form:

h(t|X)=h0(t)exp(β1X1+β2X2+ . . . +βpXp)=h0(t)exp(β′X).

This instantaneous hazard function gives the hazard time t for an individual with covariate p-vector (p explanatory variables; herein gene/protein expression levels) X. The baseline hazard, h0(t), is common to all the individuals (herein it is determined on the cohort of HGG patients under study). The expression exp(β′X) is a regression model of a multiplicative combination of p covariates (X) weighted by a p-vector of regression coefficients (′). Herein, these regression coefficients are specific of the cohort studied, the genomic level studied (transcriptome or proteome) and the technology used to measure expression levels. They must be calculated for each combination of these parameters. For example, in certain embodiments, the gene expression risk scores are calculated according to the following Cox risk equation:

(0.587×CHAF1B)+(0.326×PDLIM4)+(−0.470×EDNRB)+(0.532×HJURP).

An aggressiveness HGG grading and/or HGG survival outcome expression profile map may further contain information about methylation status of the MGMT promoter and/or mutational status of IDH1.

Comparison of an expression pattern obtained for a biological sample of a HGG patient against an expression profile map established for a particular HGG aggressiveness and/or HGG survival outcome may comprise comparison of the normalized expression levels on a biomarker-by-biomarker basis and/or comparison of ratios of expression levels within the set of biomarkers or yet comparison of the gene expression risk scores calculated from the normalized expression levels.

Based on the results of the comparison, a prognosis may be provided. The term “providing a prognosis” is used herein to mean providing information regarding the impact of the presence of HGG on a patient's future health. Providing a prognosis may include predicting one or more of: HGG progression, HGG aggressiveness, the likelihood of HGG-attributable death, the average life expectancy of the patient, and the likelihood that the patient will survive for a given amount of time (e.g., 6 months, 1 year, 2 years, 3 years, 5 years, etc).

### Selection of Appropriate Treatment

Using methods described herein, skilled physicians may select and prescribe treatments adapted to each individual patient based on the disease staging provided to the patient through determination of the expression levels of the inventive biomarkers. In particular, the present invention provides physicians with a non-subjective means to classify HGG and determine which patients may benefit from an aggressive treatment, and which patients may be spared unnecessary interventions. Selection of an appropriate therapeutic regimen for a given patient may be made based solely on the grading provided by the inventive methods. Alternatively, the physician may also consider other clinical or pathological parameter used in existing methods to grade HGG and assess its advancement.

### Treatment Monitoring and Assessment

The methods of the invention may also be used for monitoring and assessing the effects of a treatment administered to a HGG patient. For example, an expression profile of biomarkers or a biomarker combination of the invention may be determined before a treatment has been administered to a HGG patient, and compared to the expression profile of the same biomarkers or biomarker combination after a treatment has been administered to the patient.

### III—Kits

In another aspect, the present invention provides kits comprising materials useful for carrying out the grading/prognosis methods of the invention. The grading and prognosis procedures described herein may be performed by diagnostic laboratories, experimental laboratories, and practitioners. The invention provides kits that can be used in these different settings.

Materials and reagents for characterizing biological samples from HGG patients, grading HGG in patients and/or predicting survival outcome in HGG patients may be assembled together in a kit. In certain embodiments, an inventive kit comprises reagents that specifically detect expression levels of the biomarkers or biomarker combinations of the invention. Thus, in certain embodiments, a kit comprises reagents that specifically detect the expression levels of the four genes: EDNRB, HJURP, CHAF1B and PDLIM4 at the transcriptome or proteome level. In other embodiments, a kit comprises a reagent that specifically detects the expression level of at least one of the four proteins: p60/CAF-1, PDLI4, EDN/RB and HJURP. In yet other embodiments, a kit comprises reagents that specifically detect the expression levels of the three proteins: p60/CAF-1, EDN/RB and HJURP.

A kit may further comprise instructions for using the kit according to a method of the invention. Each kit may preferably comprise the reagents that render the procedure specific. Thus, for detecting/quantifying protein biomarkers (or analogs or fragments thereof), the reagents that specifically detect protein expression levels may be antibodies that specifically bind to the protein biomarkers. For detecting/quantifying the nucleic acid biomarkers, the reagents that specifically detect gene or mRNA expression levels may be nucleic acid probes complementary to the polynucleotide sequences (e.g., cDNAs or oligonucleotides) or nucleic acid primers. The nucleic acid probes may or may not be immobilized on a substrate surface (e.g., an array).

In addition, an inventive kit may further comprise at least one reagent for the detection of a protein biomarker-antibody complex formed between an antibody included in the kit and a protein biomarker present in a biological sample obtained from a patient. Such a reagent may be, for example, a labeled antibody that specifically recognizes antibodies from the species tested (e.g., an anti-human IgG), as described above. If the antibodies are provided attached to the surface of an array, a kit of the invention may comprise only one reagent for the detection of biomarker-antibody complexes (e.g., a fluorescently-labeled anti-human antibody).

Depending on the procedure, the kit may further comprise one or more of: extraction buffer and/or reagents, amplification buffer and/or reagents, hybridization buffer and/or reagents, immunodetection buffer and/or reagents, labeling buffer and/or reagents, and detection means. Protocols for using these buffers and reagents to perform different steps of the procedure may be included in the kit. The kit may further comprise one or more reagents for the determination of the methylation status of the MGMT promoter and/or the mutational status of IDH1.

The reagents may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, the kits of the present invention further comprise control samples. In other embodiments, the inventive kits comprise at least one expression profile map for HGG progression or grading and/or HGG survival outcome as described herein for use as comparison template. Preferably, the expression profile map is digital information stored in a computer-readable medium.

Instructions for using the kit according to a method of the invention may comprise instructions for processing the biological sample obtained from the HGG patient, instructions for performing the test, and/or instructions for interpreting the results as well as a notice in the form prescribed by a governmental agency (e.g., FDA) regulating the manufacture, use or sale of pharmaceuticals or biological products.

### IV—Screening of Candidate Compounds or Treatment Assessment

As noted above, the inventive biomarkers whose expression profiles correlate with HGG progression/grading and/or survival outcome are attractive targets for the identification of new therapeutic agents (e.g., using screens to detect compounds or substances that reduce or inhibit the expression of these biomarkers).

Accordingly, the present invention provides methods for the identification of compounds potentially useful for preventing or slowing the progression of HGG and increasing the survival of HGG patients.

An inventive method of screening comprises incubating a biological system, which expresses the inventive biomarkers, with a candidate compound under conditions and for a time sufficient for the candidate compound to modulate the expression of the biomarkers, thereby obtaining a test system; incubating the biological system under the same conditions and for the same time absent the candidate compound, thereby obtaining a control system; measuring the expression levels of the biomarkers in the test system; measuring the expression level of the biomarkers in the control system; and determining that the candidate compound modulates the expression of the biomarker if the expression levels measured in the test sample are lower than or greater than the expression levels measured in the control sample.

As already mentioned above and demonstrated in the Examples section, the Applicants have found that over-expression of EDNRB correlates with better prognosis in terms of survival outcome in HGG patients while over-expression of EDN/RB was found to be associated with a pejorative evaluation of HGGs. They have also found that over-expression of HJURP, CHAF1B, and/or PDLIM4 at the transcriptome and proteome levels, is associated with higher grade of HGGs and shorter survival outcome in HGG patients. Consequently, candidate compounds that are potentially useful for preventing or slowing the progression of HGG and/or for improving the survival outcome in HGG patients are compounds that induce over-expression of EDNRB and inhibit the over-expression of HJURP, CHAF1B, and/or PDLIM4; or compounds that inhibit over-expression of p60/CAF-1, PDLI4, EDN/RB and HJURP, or compounds that inhibit the over-expression of p60/CAF-1, EDN/RB and HJURP; or compounds that inhibit the over-expression of at least one of the proteins: p60/CAF-1, PDLI4, EDN/RB and HJURP.

Biological Systems. The screening methods of the present invention may be carried out using any type of biological systems, e.g., a cell, a biological fluid, a biological tissue, or an animal. In certain embodiments, the methods are carried out using a system that can exhibit HGG (e.g., an animal model). In other embodiments, the methods are carried out using a biological entity that expresses or comprises the biomarkers of the invention (e.g., a cell or tissue).

In certain preferred embodiments, the screening methods of the present invention are carried out using cells that can be grown in standard tissue culture plastic ware. Such cells include all appropriate normal and transformed cells derived from any recognized sources. Preferably, cells are of mammalian (human or animal such as rodent or simian) origin. More preferably, cells are of human origin. Mammalian cells may be of any organ or tissue (e.g., brain, bone marrow or cerebrospinal fluid) and of any cell types as long as the cells express the biomarkers of the invention.

Cells to be used in the practice of the methods of the present invention may be primary cells, secondary cells, or immortalized cells (e.g., established cell lines). They may be prepared by techniques well known in the art (for example, cells may be isolated from brain, bone marrow, or cerebrospinal fluid) or purchased from immunological and microbiological commercial sources (for example, from the American Type Culture Collection, Manassas, Va.). Alternatively or additionally, cells may be genetically engineered to contain, for examples, genes of interest (in particular the four gene biomarkers of the invention).

Selection of a particular cell type and/or cell line to perform an assay according to the present invention will be governed by several factors including, in particular, the intended purpose of the assay. For example, an assay developed for primary drug screening (i.e., first round(s) of screening) is preferably performed using established cell lines, which are commercially available and usually relatively easy to grow, while an assay to be performed later in the drug development process is preferably performed using primary and secondary cells, which are generally more difficult to obtain, maintain and/or grow than immortalized cells but which represent better experimental models for in vivo situation.

Examples of established cell lines that can be used in the practice of the screening methods of the present invention include human glioblastoma cell lines, human glioblastoma-astrocytoma, epithelial-like cell lines, and human glioma cell lines. Primary and secondary cells that can be used in the inventive screening methods include, but are not limited to, astrocytes, oligoastrocytomas, and oligodendrocytes.

Cells to be used in the inventive assays may be cultured according to standard cell culture techniques. For example, cells are often grown in a suitable vessel in a sterile environment at 37° C. in an incubator containing a humidified 95% air-5% CO2 atmosphere. Vessels may contain stirred or stationary cultures. Various cell culture media may be used including media containing undefined biological fluids such as fetal calf serum. Cell culture techniques are well known in the art and established protocols are available for the culture of diverse cell types (see, for example, R. I. Freshney, “Culture of Animal Cells: A Manual of Basic Technique”, 2nd Edition, 1987, Alan R. Liss, Inc.).

In certain embodiments, the screening methods are performed using cells containing in a plurality of wells of a multi-well assay plate. Such assay plates are commercially available, for example, from Stratagene Corp. (La Jolla, Calif.) and Corning Inc. (Acton, Mass.) and include, for example, 48-well, 96-well, 384-well and 1536-well plates.

Candidate Compounds. As will be appreciated by those of ordinary skill in the art, any kind of compounds or agents can be tested using the inventive methods. A candidate compound may be a synthetic or natural compound; it may be a single molecule or a mixture or a complex of different molecules. In certain embodiments, the inventive methods are used for testing one or more compounds. In other embodiments, the inventive methods are used for screening collections or libraries of compounds. As used herein, the term “collection” refers to any set of compounds, molecules or agents, while the term “library” refers to any set of compounds, molecules or agents that are structural analogs.

Collections of natural compounds in the form of bacterial, fungal, plant and animal extracts are available from, for example, Pan Laboratories (Bothell, Wash.) or MycoSearch (Durham, N.C.). Libraries of candidate compounds that can be screened using the methods of the present invention may be either prepared or purchased from a number of companies. Synthetic compound libraries are commercially available from, for example, Comgenex (Princeton, N.J.), Brandon Associates (Merrimack, N.H.), Microsource (New Milford, Conn.), and Aldrich (Milwaukee, Wis.). Libraries of candidate compounds have also been developed by and are commercially available from large chemical companies, including, for example, Merck, Glaxo Welcome, Bristol-Meyers-Squibb, Novartis, Monsanto/Searle, and Pharmacia UpJohn. Additionally, natural collections, synthetically produced libraries and compounds are readily modified through conventional chemical, physical, and biochemical means. Chemical libraries are relatively easy to prepare by traditional automated synthesis, PCR, cloning or proprietary synthetic methods (see, for example, S. H. DeWitt et al., Proc. Natl. Acad. Sci. U.S.A. 1993, 90:6909-6913; R. N. Zuckermann et al., J. Med. Chem. 1994, 37: 2678-2685; Carell et al., Angew. Chem. Int. Ed. Engl. 1994, 33: 2059-2060; P. L. Myers, Curr. Opin. Biotechnol. 1997, 8: 701-707).

Useful agent for the treatment of HGGs may be found within a large variety of classes of chemicals, including heterocycles, peptides, saccharides, steroids, and the like. In certain embodiments, the screening methods of the invention are used for identifying compounds or agents that are small molecules (i.e., compounds or agent with a molecular weight<600-700 Da).

The screening of libraries according to the inventive methods will provide “hits” or “leads”, i.e., compounds that possess a desired but not-optimized biological activity. The next step in the development of useful drug candidates is usually analyzing the relationship between the chemical structure of a hit compound and its biological or pharmacological activity. Molecular structure and biological activity are correlated by observing the results of systemic structural modification on defined biological end-points. Structure-activity relationship information available from the first round of screening can then be used to generate small secondary libraries, which are subsequently screened for compounds with higher affinity. The process of performing synthetic modifications of a biologically active compound to fulfill all stereoelectronic, physicochemical, pharmacokinetic, and toxicologic factors required for clinical usefulness is called lead optimization.

Candidate compounds identified as potential HGG therapeutic agents by screening methods of the present invention can similarly be subjected to a structure-activity relationship analysis, and chemically modified to provide improved drug candidates. The present invention also encompasses these improved drug candidates, as well as pharmaceutical compositions thereof.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Some of the results reported presented below were described in two scientific papers (de Tayrac et al., Clin. Cancer Res., January 2011, 17: 317-327; and Saikali et al., “Prognostic significance of EDN/RB, HJURP, p60/CAF-1 and PDLI4, four new markers in high-grade gliomas”, submitted to review). The contents of the scientific papers are included herein by reference in their entirety, including the supplemental information and figures.

### Example 1

### A Four-Gene Signature Associated with Clinical Outcome in High-Grade Gliomas

### Materials and Methods

Study Samples. The local cohort comprised a total of 194 patients with newly diagnosed and untreated high grade gliomas (HGGs) admitted to the University hospitals involved in the French Cancérôpole Grand-Ouest Glioma Project. Patients were selected retrospectively during the period from 1998 to 2008 with a follow-up time of a minimum of 2 years. Tumor samples were collected in accordance with the French regulations and the Declaration of Helsinki. All patients gave their informed consent before inclusion. Initial histology was confirmed by a central review involving at least two neuropathologists according to the WHO classification of central nervous system tumors (Louis et al., Acta Neuropathol., 2007, 114(2): 97-109). Patient characteristics are summarized in Table 1. Total DNAs and RNAs were isolated from frozen samples of primary brain tumors stored (−80° C.) at the Cancéropôle Biological Resource Centers. Quality of DNA samples was assessed on 1% agarose gel and RNA integrity was confirmed using the Agilent 2100 Bioanalyzer (Agilent Technologies).

TABLE 1
Patients' clinical characteristics and stratification
on the 4-gene expression risk score.

| | | Patients with | Patients with |
|---|---|---|---|
| | All Patients | low risk score | high risk score |
| Characteristics | (N = 194) | (N = 55) | (N = 139)) |
| Age, y | | | |
| Median | 57 | 52 | 58 |
| Range | 13-80 | 13-77 | 16-80 |
| Age, n (%) | | | |
| ≦50 y | 64 (33) | 25 (46) | 39 (28) |
| ≧50 y | 130 (67) | 30 (54) | 100 (72) |
| Univariate analysis | P = 0.006 | | |
| Sex, n (%) | | | |
| Male | 103 (53) | 32 (58) | 71 (51) |
| Female | 91 (47) | 23 (42) | 68 (49) |
| Univariate analysis | P = 0.85 | | |
| Preopoerative KPS | | | |
| performance status (%) | | | |
| Median | 80 | 85 | 80 |
| Range | 20-100 | 40-100 | 20-100 |
| ND; n | 15 | 9 | 6 |
| Univariate analysis | P = 0.692 | | |
| Extent of surgery, n (%) | | | |
| None | 2 (1) | 0 (0) | 2 (1) |
| Biopsy | 13 (7) | 5 (9) | 8 (6) |
| Debulking | | | |
| Partial resection | 49 (25) | 15 (25) | 34 (25) |
| Complete resection | 123 (63) | 34 (62) | 89 (64) |
| ND | 7 (4) | 1 (1) | 6 (4) |
| Univariate analysis | P = 0.438 | | |
| RTOG RPA | | | |
| classification, n (%) | | | |
| I-II | 26 (14) | 18 (33) | 8 (6) |
| III-IV | 66 (34) | 17 (31) | 49 (35) |
| V-VI | 99 (51) | 19 (35) | 80 (58) |
| ND | 3 (2) | 1 (1) | 2 (1) |
| Univariate analysis | P < 0.001 | | |
| Therapy, n (%) | | | |
| None | 4 (2) | 1 (1) | 3 (2) |
| Radiotherapy alone | 20 (10) | 5 (9) | 15 (11) |
| Chemotherapy alone | 7 (4) | 5 (9) | 2 (1) |
| Radiotherapy plus | | | |
| chemotherapy | | | |
| Temozolomide | 106 | 18 (33) | 88 (63) |
| PCV^{a} | 28 | 19 (35) | 9 (7) |
| Other^{b} | 27 (14) | 5 (9) | 22 (16) |
| ND | 2 (2) | 2 (4) | 0 |
| Univariate analysis | P = 0.366 | | |
| IDH1 mutation, n (%) | | | |
| Mutated^{c} | 30 (15) | 20 (37) | 10 (7) |
| Wild-type | 159 (82) | 32 (58) | 127 (92) |
| ND | 5 (3) | 3 (5) | 2 (1) |
| Univariate analysis | P < 0.001 | | |
| MGMT status, n (%) | | | |
| Unmethylated | 94 (49) | 20 (37) | 74 (53) |
| Methylated | 90 (46) | 32 (58) | 58 (42) |
| ND | 10 (5) | 3 (5) | 7 (5) |
| Univariate analysis | P < 0.001 | | |
| Findings on | | | |
| pathologic review, n | | | |
| Glioblastoma^{d} | 145 | 23 | 122 |
| Anaplastic | 38 | 22 | 16 |
| astrocytoma^{e} | | | |
| With necrosis and | 25 | 13 | 12 |
| vascular proliferation | | | |
| Anaplastic | 11 | 10 | 1 |
| oligodendroglioma^{e} | | | |
| With necrosis and | 3 | 2 | 1 |
| vascular proliferation | | | |
| Univariate analysis | P < 0.001 | | |
| Survival, mo | | | |
| Median | 16.2 | 55.8 | 14.5 |
| 95% CI | 14.7-18.3 | 26.0 to NR | 12.5-16.0 |

^{a}PCV consists of three chemotherapy drugs: Procarbazine, CCNU, and Vincristine.
^{b}Other: includes topotecan, BCNU, Gemini and 8 drugs in one EORTC trial chemotherapy.
^{c}Sequencing results (not shown).
^{d}Glioblastoma included 4 secondary glioblastomas.
^{e}Anaplastic astrocytoma included oligoastrocytoma.
Abbreviation: NR, median survival not reached.

RT-qPCR Analysis. RT-qPCR reactions were performed as described previously (de Tayrac et al., Genes Chromosomes Cancer, 2009, 48(1): 55-68) with B2M (β-2 microglobulin) and HPRT1 (hypoxanthine phosphoribosyltransferase) as internal controls.

IDH1 Mutations. Exon 4 of the IDH1 gene was amplified with the use of a PCR assay and sequenced in DNA from the tumor from each patient, as described previously (Parsons et al., Science, 2008, 321(5897): 1807-12). Patients were screened for somatic mutations affecting the R132 residue of IDH1.

MGMT Promoter Methylation. The pyrosequencing methylation assay was performed with the PyroMark Q96 CpG MGMT kit (Qiagen), according to the manufacturer's protocol. Samples were considered methylated if they had average CpG methylation ≧9% and unmethylated if they had average methylation <9%, in duplicate reactions (Dunn et al., Br. J. Cancer, 2009, 101(1): 124-31).

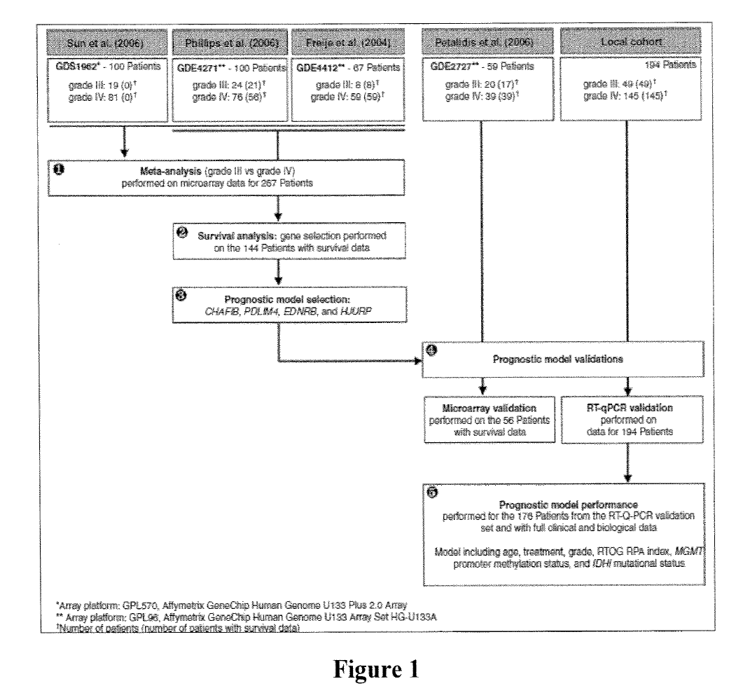

External Data Collection. External microarray data for 326 patients were collected from four Gene Expression Omnibus (GEO) HGGs datasets (Petalidis et al., Mol. Cancer. Ther., 2008, 7(5): 1013-24; Phillips et al., Cancer Cell, 2006, 9(3): 157-73, 2006; Freije et al., Cancer Res., 2004, 64(18): 6503-10; Sun et al., Cancer Cell, 2006, 9(4): 287-300). There were 22215 common probe sets in the three data sets. Baseten log-transformed intensities were centered using the scale function of the R base package. Data sets characteristics and analysis workflow are presented in FIG. 1.

Statistical Analysis.

Combined Analysis of Microarray Data. Combined analysis was performed on 267 patients (GDS1962, GSE4271 and GSE4412) using the Bioconductor RankProd package (Hong et al., Bioinformatics, 2006, 22(22): 2825-7). This package utilizes the rank product non-parametric method to identify up- and down-regulated genes between anaplastic astrocytomas and glioblastomas (Breitling et al., FEBS Lett., 2004, 573(1-3): 83-92). The RankProd package was chosen for its ability to easily combine data sets from different origins (laboratories and environments) into a single analysis. It was also shown that this non-parametric method outperforms other meta-analysis methods in terms of sensitivity and specificity (Hong et al., Bioinformatics, 2006, 22(22): 2825-7). Individual analyses were also performed for each study (two-sided Student t test) and results were combined. Genes were considered to be differentially expressed for a corrected p-value (False Discovery Rate) below 0.05 and a fold-change greater than 2 in at least one of the two approaches. Functional annotation analyses were assessed using the Database for Annotation, Visualisation, and Integrated Discovery (david.abcc.ncifcrf.gov/) and unsupervised PCA with integration of biological knowledge (de Tayrac et al., BMC Genomics, 2009, 10: 32). Benjamini corrected pvalues were used for multiple testing (P<0.05).

Survival Analysis and Prognostic Model Selection. A cross-study analysis of genes that can assist in the prognostication of survival by univariate Cox regression analysis was performed. Gene expression was used as a predictor and survival time (in months) as the response. In order to select the significant genes, the FDR was controlled with the Benjamini-Hochberg (BH) correction and set the p-value threshold at 0.01. To build an optimal survival model, survival-associated genes were selected with the rbsury R package. Briefly, this package allows a sequential selection of genes based on the Cox proportional hazard model and on maximization of log-likelihood. To increase robustness, this package also selects survival associated genes by repetition (1000 times) of a separation between the training and validation sets of samples. Regression coefficients of the optimal survival model were estimated after adjustment on the study factor. Risk scores were determined using classical Cox model risk formulae with a linear combination of the gene-expression values weighted by the estimated regression coefficients. Time-dependent ROC curve analyses were used to select the optimal risk cut-offs for the stratification of patients. The Kaplan-Meier method was used to estimate the survival distributions. Logrank tests were used to test the difference between survival groups. Analyses were carried out with the survival and survivalROC R packages.

Prognostic Model Validation and Performances. A model including clinical factors—age, treatment, histological grade and risk classes as defined by the Radiation Therapy Oncology Group (RTOG) by recursive partitioning analysis (RPA) (Curran et al., J. Natl. Cancer Inst., 1993, 85(9): 704-10)—along with MGMT methylation status and IDH1 mutational status was constructed. The discriminatory capability of the model was evaluated with the gene-expression risk-score as compared with the model without the gene-expression risk-score using C statistics. Differences in discrimination were evaluated using a non-parametric approach (DeLong et al., Biometrics, 1988, 44: 837-845). Model calibration was assessed using the Hosmer-Lemeshow Chi-square test (Hosmer and Lemeshow, “Applied logistic regression”, New York, John Wiley, 1989). Analyses were performed using the Hmisc and Design R packages.

### Results

Data sets characteristics and analysis workflow are summarized in FIG. 1.

Consensus Gene Selection in High-Grade Gliomas. Combined analysis and individual study approaches were performed to define a consensus gene expression signature in HGGs that could be used to find biomarkers associated with clinical outcomes. This signature was composed of 438 gene probe sets with 65 identified by both approaches. Associated enriched GO processes were related to invasion, angiogenesis, response to stress, and morphogenesis. Among the consensus genes strongly associated with grading, the nine genes (CHI3L1, ADAM12, S100A4, TIMP1, NDRG2, NTRS2, LUZP2, ALDH5A1 and RASL10A) were selected and validated by RT-qPCR (P<0.001) on a subset of 90 HGG samples.

A Gene Expression Risk-Score Associated with Survival In High-Grade Gliomas. To assess the survival prognosis capabilities of the 438 selected probe sets, univariate Cox analyses of the expression data for these genes were performed, with overall survival (OS) as a dependent variable. The genes were ranked on the basis of their predictive power (univariate z score). The genes having a highly significant association with survival were then selected and identified 40 genes with high predictive power were identified. According to the univariate z score, 26 were risk genes and 14 were protective genes. Risk genes were related to GO biological process “cell cycle” (CDC25A, ASPM, CHAF1B, CENPE, CEP55, CDC20, NCAPG, AURKA) and to “ECM-receptor interaction and Focal Adhesion KEGG” pathways (HMMR, COL1A2, COL4A2, COL1A1, COL4A1, MET). Interestingly, five of the protective genes were related to GO biological process “nervous system development” (EDNRB, ABLIM1, ALDH5A1, NDRG2, FGF12).

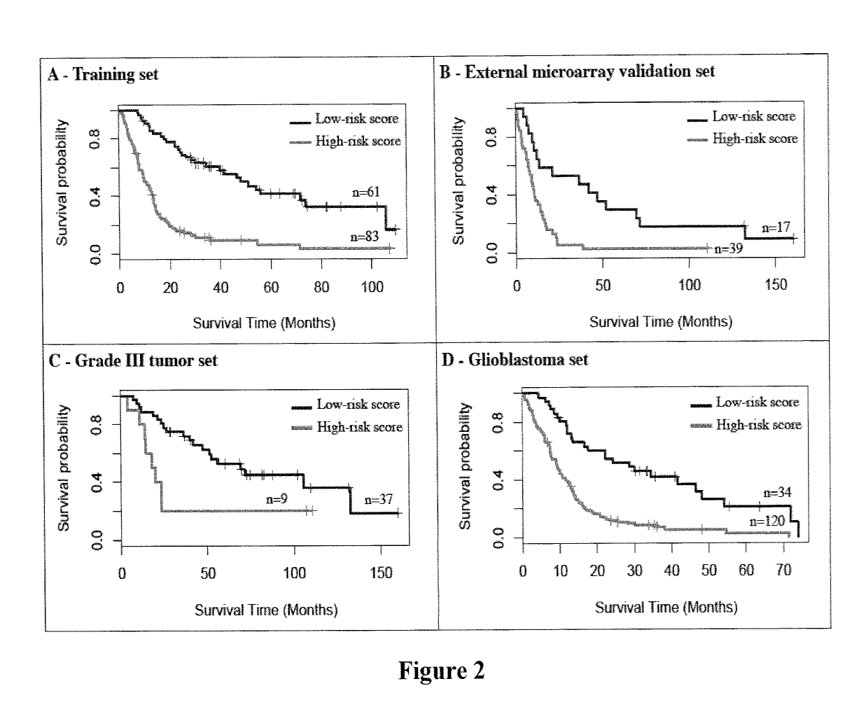

Multivariate Cox regression analyses were performed to create an optimal gene-based survival model. The 40 selected genes were used to sequentially construct survival models. The model best associated with survival (P<0.001) and with good discrimination ability (C statistic, 0.843; 95% CI, 0.647-0.827) was based on the expression of four genes: CHAF1B, PDLIM4, EDNRB and HJURP. The relative contributions of each of the four genes in the multivariate analysis are summarized in the portion of the Cox risk equation that captures the individual risk profile: (0.587×CHAF1B)+(0.326×PDLIM4)+(−0.470×EDNRB)+(0.532×HJURP). Patients were ranked according to their risk score. The optimal risk cut-off was assessed and used for the stratification of patients into two groups: low risk of death and high risk of death. Patients with a low-risk of death (25 anaplastic astrocytomas and 36 glioblastomas) had a median OS of 46.6 months (95% CI, 28.7-73.9), which was significantly longer than 11.7 months (95% CI, 9.0-13.5) for patients with a high risk of death (4 anaplastic astrocytomas and 79 glioblastomas), P<0.001 by the log-rank test (FIG. 2A).

During the present work, the MD Anderson group published a nine-gene panel (AQP1, CHI3L1, EMP3, GPNMB, IGFBP2, LGALS3, OLIG2, PDPN and RTN1) to predict outcome in glioblastoma (Colman et al., Neuro-Oncology, 2009, 12(1): 49-57). Six of these genes were also found in the present consensus gene selection. The present four-gene panel was compared to the MD Anderson group nine-gene predictor. Both models were highly significant (P=1e−08 and P=3e−05, respectively). The discrimination of the four-gene model was significantly higher than the discrimination of the nine-gene model (C statistic, 0.80 [95% CI, 0.72-0.86] vs. 0.76 [95% CI, 0.64-0.81], P<0.001, respectively), showing the relevance and superiority of the four-gene panel.

An external validation of the four-gene survival model was performed with an independent microarray data set comprising 56 HGGs with survival data reported by Petalidis et al. (Mol Cancer Ther., 2008, 7(5): 1013-2). Patients were divided into two groups on the basis of the four-gene model (low or high risk of death). The low-risk group was composed of 12 anaplastic astrocytomas and 5 glioblastomas and the high risk of 5 anaplastic astrocytomas and 34 glioblastomas. The OS was higher in low-risk HGGs compared to high-risk HGGs (17.8 months [95% CI, 9.6-47.9] vs. 9.3 months [95% CI, 7.2-13.9], respectively; P<0.001; FIG. 2B). The discrimination was as good as in the original data (C statistic, 0.852; 95% CI, 0.673-0.933).

Model validation was also performed to determine if the four-gene expression data contained survival-predictive information that was distinct from the prediction embedded within histologic grade. In the whole anaplastic astrocytoma set, the OS was higher in low-risk patients (n=9) compared to high-risk patients (n=37) (69.4 months [95% CI, 41.8 to not reached] vs. 19.7 months [95% CI, 13.7 to not reached], respectively; P<0.05; FIG. 2C). In the whole glioblastoma set, low-risk patients (n=34) had a much higher OS (30.07 months; 95% CI, 17.7-54.2) compared to high-risk patients (n=120; 9.3 months; 95% CI, 7.6-11.7; P<0.001; FIG. 2D).

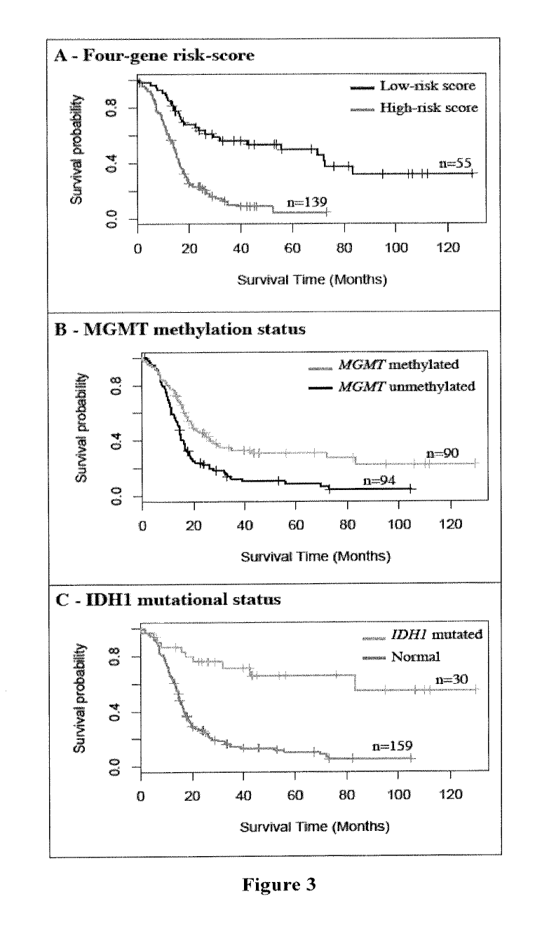

Evaluation of the Gene Expression Risk-Score Performances. A cohort of 194 patients with extensive bio-clinical parameters was used to validate the performances of the four-gene classifier (Table 1). Univariate analyses showed that the gene expression risk-score, the DNA methylation status of the MGMT promoter, and the IDH1 mutational status were significantly associated with the OS in this cohort. In the whole cohort, patients were divided into two groups on the basis of the risk-score model with log2-transformed data issued from RT-qPCR analysis. The OS was clearly higher for low-risk patients (55.8 months; 95% CI, 26.0 to not reached) compared to high-risk patients (14.5 months; 95% CI, 12.5 to 16.0; P<0.001; as shown in FIG. 3A). In this population, MGMT-methylated tumors, compared to unmethylated tumors, had a significantly better OS (19.5 months [95% CI, 16.7 to 29.4] vs. 14.5 months [95% CI, 11.4 to 16.2], respectively; P<0.001; FIG. 3B). Similarly, in this group, IDH1-mutated tumors had a much higher OS (median survival not reached; 95% CI, 42.5 to not reached) than IDH1-nonmutated tumors (14.9 months; 95% CI, 13.7 to 16.5; P<0.001; FIG. 3C).

Two multivariate models were built, both including age, treatment, grade, RTOG RPA classes, MGMT methylation status and IDH1 mutational status; one with and one without the four-gene expression risk-score. These models were used to estimate the prognostic value of the gene expression risk-score (i) for 176 of the 194 patients with complete data for all variables and (ii) for a subset of patients treated with temozolomide chemoradiation (n=105). Results are provided in Table 2. In both cases, the gene expression risk-score was strongly associated with survival (hazard ratio=0.49; 95% CI, 0.30-0.81; P=0.005; and hazard ratio=0.37; 95% CI, 0.18-0.77; P=0.008, respectively) and all models showed excellent discrimination, with C statistics over 0.80. In the whole cohort and for the patients treated with temozolomide chemotherapy, the C statistic improved significantly with the addition of the gene expression risk-score in the model (0.816 vs. 0.846, P<0.001 and 0.792 vs. 0.822, P<0.001, respectively), showing that the four-gene risk-score added beyond standard clinical parameters and beyond both the MGMT methylation status and the IDH1 mutational status.

TABLE 3
Comparison of prognostic model adjusted for clinical factors along with MGMT promoter
methylation status and IDH1 mutational status, with or without the 4-gene risk score.

| | Prediction Model | |
|---|---|---|
| | Without the 4-gene | With the 4-gene |
| | expression risk score | expression risk score |
| Whole cohort (n = 176) | | |
| Age <50 y vs >50 y | | |
| Hazard ratio (95% CI) [P] | 0.99 (0.97-1.01) [0.47] | 0.99 (0.97-1.01) [0.56] |
| RTOG RPA classification, per unit increase | | |
| Hazard ratio (95% CI) [P] | 1.05 (0.71-1.59) [0.78] | 1.02 (0.68-1.53) [0.93] |
| Treatment, per unit increase | | |
| Hazard ratio (95% CI) [P] | 0.81 (0.66-0989) [0.03] | 0.83 (0.69-1.01) [0.07] |
| Histology, grade IV vs III | | |
| Hazard ratio (95% CI) [P] | 3.28 (1.74-6.14) [<0.001] | 1.62 (0.84-3.13) [0.01 |
| MGMT methylated vs unmethylated | | |
| Hazard ratio (95% CI) [P] | 0.61 (0.43-0.87) [0.007] | 0.61 (0.42-0.88) [0.007] |
| IDH1 mutated vs unmutated | | |
| Hazard ratio (95% CI) [P] | 0.32 (0.14-0.71) [0.005] | 0.38 (0.17-0.84) [0.02] |
| Four-gene risk score, low vs high | | |
| Hazard ratio (95% CI) [P] | — | 0.49 (0.30-0.81) [0.005] |
| Discriminatory capability | | |
| C statistic (95% CI) | 0.816 (0.739-0.891) [<0.001] | 0.846 (0.770-0913) |
| [P value for difference] | | |
| Accuracy of calibration at 3 y | | |
| χ² [P value for difference] | 3.61 [0.935] | |
| Patients treated with temozolomide | | |
| chemoradiation (n = 105) | | |
| Age <50 y vs >50 y | | |
| Hazard ratio (95% CI) [P] | 1.00 (0.97-1.03) [0.97] | 1.00 (0.97-1.03) [0.98] |
| RTOG RPA classification, per unit increase | | |
| Hazard ratio (95% CI) [P] | 1.22 (0.58-2.61) [0.59] | 1.34 (0.66-2.80) [0.43] |
| Histology, grade IV vs III | | |
| Hazard ratio (95% CI) [P] | 1.67 (0.49-5.60) [0.41] | 1.06 (0.30-3.75) [0.92] |
| MGMT methylated vs unmethylated | | |
| Hazard ratio (95% CI) [P] | 0.60 (0.37-0.95) [0.03] | 0.53 (0.33-0.86) [0.01] |
| IDH1 mutated vs unmutated | | |
| Hazard ratio (95% CI) [P] | 0.10 (0.01-0.77) [0.03] | 0.11 (0.01-0.89) [0.04] |
| Four-gene risk score, low vs high | | |
| Hazard ratio (95% CI) [P] | — | 0.37 (0.18-0.78) [0.008] |
| Discriminatory capability | | |
| C statistic (95% CI) | 0.793 (0.592-0.937) [<0.001] | 0.821 (0.688-0903) |
| [P value for difference] | | |
| Accuracy of calibration at 3 y | | |
| χ² [P value for difference] | 3.55 [0.939] | 3.58 [0.937] |

The performance of the gene expression risk-score was also evaluated on a subset of 98 patients with glioblastoma who underwent tumor resection and who were treated with radiotherapy plus concomitant and adjuvant temozolomide. After adjustment for RTOG RPA classes and MGMT promoter methylation status, multivariate analysis confirmed that the four-gene expression riskscore was an independent marker robustly associated with outcome for glioblastoma patients treated with standard protocol (hazard ratio=0.386, 95% CI, 0.164 to 0.910, P value=0.03).

### Discussion

Molecular studies of HGGs have highlighted the heterogeneity of these tumors, and have linked molecular signatures to their natural history and to differences in survival rates. It is likely that the ability to identify such molecular subtypes of tumors will be essential for guiding therapeutic advances. In this study, a risk-score model based on the expression of four genes for the stratification of patients with HGGs is reported. This risk calculation is based on a consensus gene expression signature and is strongly associated with survival independently from current clinical risk factors, IDH1 mutational status and MGMT promoter methylation status. The initial step of the present study consisted in a discovery phase for the identification of biomarkers repeatedly correlated with both tumor aggressiveness and patient outcome. It should be noticed that information regarding the therapeutic regimens was not incorporated in the meta-analysis of microarray data sets. While this could have weakened this discovery phase, combining multiple and independent data sets was also an asset to identify robust biomarkers. Moreover, the RTqPCR validation of the four-gene signature in an external cohort of patients showed that the two risk groups had significant differences in OS independently from treatment. These results suggest that the four genes are relevant molecular markers in HGGs.

One explanation for the association between the four-gene signature and clinical outcome could be that it may detect the molecular fingerprints inherent to glioma aggressiveness. The proposed multimarker panel is based on the expression of EDNRB, CHAF1B, PDLIM4, and HJURP. In this model, the over-expression of EDNRB correlates with better prognosis. EDNRB encodes the endothelin receptor type B implicated in tumor proliferation, survival, invasion, angiogenesis and metastasis (Nelson et al., Nat. Rev. Cancer, 2003, 3(2): 110-6). Freije et al. (Cancer Res., 2004, 64(18): 6503-10) have reported EDNRB as a member of the neurogenesis related genes group that portends the longest survival. The three other genes of our model (CHAF1B, PDLIM4, HJURP) are correlated with a higher risk of death. CHAF1B encodes the p60 subunit of the chromatin assembly factor I (CAF-I), which plays a major role in chromatin assembly after replication and DNA repair. It has been proposed as a specific marker of actively proliferating cells (Polo et al., Cancer Res., 2004, 64(7): 2371-81) and as a predictor of poor outcome in squamous cell carcinoma of the tongue (Staibano et al., Histopathology, 2007, 50(7): 911-9). PDLIM4, a LIM domain gene also known as RIL, is suspected to have tumor suppressor functions in myeloid diseases (Boumber et al., Cancer Res., 2007, 67(5): 1997-2005) and prostate cancer (Vanaja et al., Clin. Cancer Res., 2006, 12(4): 1128-36) by either LOH, deletion or hypermethylation. However, its extreme up-regulation by integrin-promoted demethylation has been recently reported (Chen et al., J. Biol. Chem., 2009, 284(3): 1484-94) in breast carcinoma cells together with other genes also validated in the present study (S100A4, NCAPG), suggesting a potential oncogenic function of PDLIM4. The Holliday Junction Recognition Protein (HJURP) was recently shown to be an indispensable factor for cell-cycle-regulation of centromeric chromatin assembly (Foltz et al., Cell, 2009, 137(3): 472-84; Dunleavy et al., Cell, 2009, 137(3): 485-97) and for chromosomal stability in immortalized cancer cells (Kato et al., Cancer Res., 2007, 67(18): 8544-53). It has also recently been suggested that HJURP could be implicated in glioma malignancy (Valente et al., BMC Mol. Biol., 2007, 10(1): 17). These studies and the present findings suggest that these four genes are important molecular components of astrocytic tumors aggressiveness.

The two risk groups defined by the four-gene classifier are also characterized by the expression change of genes related to cancer malignancy or survival of gliomas. Genes highly expressed in high-risk HGGs are remarkably related to cell cycle and cytokinesis, in accordance with the fact that aggressive tumors exhibit a high percentage of cycling cells. This was also reported for the Proliferative subgroup of HGGs identified by Philips et al. (Cancer Cell, 2006, 9(3): 157-73). Most of the genes highly expressed in low-risk HGGs are related to the development of the nervous system. Other authors (Phillips et al., Cancer Cell, 2006, 9(3): 157-73; Freije et al., Cancer Res., 2004, 64(18): 6503-10; Shirahata et al., Cancer Sci., 2009, 100(1): 165-7) also described a correlation between neuronal markers and the favorable subclasses of HGGs. These findings underline that the two risk groups have distinct molecular phenotypes and suggest that they may respond differently to therapeutic regimens. Multivariate analysis confirmed that both the mutations of IDH1 and the presence of MGMT promoter methylation were associated with a survival benefit in the whole cohort of HGGs and in the subgroup of patients with glioblastoma treated similarly with temozolomide chemoradiation. This analysis also showed that the four-gene expression risk-score was strongly associated with outcome, independently from clinical and molecular risk factors. The performance evaluation indicated that the four-gene added beyond the prognostic capabilities of all these factors. These results suggest that the four-gene status, along with the existing clinical and other molecular markers, could be used to optimize patient stratification.

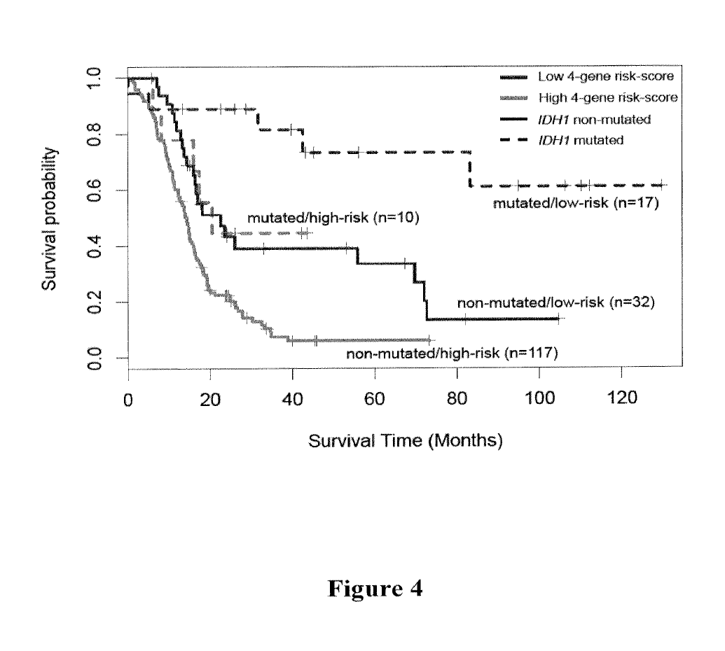

As an illustration, when combined with the IDH1 mutational status, the four-gene risk-score allowed the identification of three groups of HGGs (good-, intermediate- and poor-outcome groups) with significant differences in OS (P<0.001, FIG. 4). The group of HGGs with intermediate-outcome (non-mutated/low-risk or mutated/high-risk) was characterized by a median survival of 20.6 months (95% CI, 16.5 to 72.1), as compared to 14 months (95% CI, 12.3 to 15.2) for the poor-outcome group (nonmutated/high-risk) and to a median survival not reached (95% CI, 83.2 to not reached) for the good-outcome group (mutated/low-risk). For this intermediate-outcome group (representing 24% of the whole cohort), the MGMT methylation status did not provide any predictive information (P=0.5) and the median survival time was similar to that of patients with methylated MGMT promoter. These results suggest the importance of using the four-gene signature as a stratification factor for the design of future comparative therapeutic trials.

### Example 2

### A Four-Protein Signature Associated with Clinical Outcome in High-Grade Gliomas

### Materials and Methods

Patients and Tissue Specimens. This study was conducted on a total of 96 consecutive patients, who were hospitalized in the Neurosurgical Department of the Rennes University Hospital for surgical procedures of histologically diagnosed HGG from 1999 to 2006. Tumor samples were collected in accordance with the French regulations and the Declaration of Helsinki. All initial histological specimens were reviewed by a single neuropathologist (blinded on the patient's data) for confirmation of the original diagnosis according to the WHO classification of central nervous system tumors (Louis et al., Acta Neuropathol., 2007, 114(2): 97-109). Clinical data systematically included age at the diagnosis, gender and preoperative performance status. All patients had brain MRI (without and with gadolinium) performed before and 72 hours after surgery. Patients underwent a subtotal or a gross total resection. Total excision was retained when no residual enhancement was seen on post-operative control MRI. Survival time was measured from the date of surgery until death or last clinical examination updated to Jul. 1, 2009. No patients developed any leptomeningeal dissemination or distant metastasis. Clinical information is detailed in Table 3. Six autopsic adult normal brain tissues were obtained by collecting donations from individuals who died of non-neurological disease.

Immunohistochemical Procedure. Immunohistochemistry was performed on formalin-fixed and paraffin-embedded gliomas, using 4-μm sections. After routine deparaffinization, rehydration and blocking of endogenous peroxidase activity, antigen retrieval was performed by immersion in 0.01 M sodium citrate buffer (pH 6.0) for 40 minutes in a 80° C. water-bath. Endogenous peroxidase activity was quenched with 10% H2O2 in PBS for 20 minutes. The monoclonal mouse anti-human clone SS 53 (abcam), and clone 8Z11 (IBL) antibodies were used respectively to study p60/CAF-1 and EDN/RB expression. The monoclonal rabbit anti-human product number HPA011912 (Sigma), and product number HPA008436 (Sigma) antibodies were used respectively to study PDLI4 and HJURP expression. Primary antibodies were diluted in PBS/10% serum and applied to the sections in a humid chamber overnight at 4° C. (dilutions of 1:500, 1:50, 1:500, 1:100 for p60/CAF-1, PDLI4, HJURP and EDN/RB respectively, in antibody diluent of the Dako Cytomation kit (Trappes, France)). Tumor sections were stained using the Vectastain kit (Vector, Burlingame, USA) and biotinylated using the RTU Vectastain Elite ABC kit (Vector) according to the manufacturer's instructions. Sections were revealed using the peroxidase substrate kit (Vector) and counterstained with hematoxylin.

TABLE 3
Clinical Characteristics of the Patients
and Univariate Survival Analysis

| | | Survival |
|---|---|---|
| | All Patients | Univariate |
| Characteristic | (N = 96) | analysis |
| Age--no. | | p = 0.03 |
| ≦50 yr | 28 | |
| >50 yr | 68 | |
| Gender--no. | | NS |
| Male | 51 | |
| Female | 45 | |
| Preoperative KPS | | NS |
| performance status (%) | | |
| Median | 80 | |
| Range | 40-100 | |
| ND - no. | 6 | |
| Extent of surgery - no. | | NS |
| Biopsy | 7 | |
| Debulking | | |
| Partial resection | 18 | |
| Complete resection | 67 | |
| ND | 4 | |
| Therapy (*) - no. | | p = 0.01 |
| None | 2 | |
| Radiotherapy alone | 16 | |
| Chemotherapy alone | 3 | |
| Radiotherapy plus chemotherapy | | |
| Temozolomide | 35 | |
| PCV | 16 | |
| Other | 22 | |
| ND | 2 | |
| Findings on | | |
| pathological review - no. | | |
| Glioblastoma | 64 | |
| Anaplastic astrocytoma (**) | 24 | |
| Anaplastic oligodendroglioma | 8 | |
| Cytoplasmic EDNRB - (%) | | p = 0.0008 |
| Median | 81 | |
| Range | 12--100 | |
| Nuclear p60/CAF-1 - (%) | | p = 0.0001 |
| Median | 25 | |
| Range | 4--60 | |
| Nuclear HJURP - (%) | | p = 0.002 |
| Median | 10 | |
| Range | 0--34 | |
| Cytoplasmic PDLI4 - (%) | | p = 0.08 |
| Median | 50 | |
| Range | 4--91 | |
| Survival -- mo | | |
| Median | 16 | |
| 95CI | 14-19.1 | |

(*) PCV consists of three chemotherapy drugs: Procarbazine, CCNU and Vincristine. Other: includes topotecan, BCNU, Gemini and 8 drugs chemotherapy
(**) Anaplastic astrocytoma included oligoastrocytoma.

Control Materials. External positive controls were used for each staining: breast adenocarcinoma for p60/CAF-1, normal striated muscle for PDLI4, normal liver for HJURP and lung adenocarcinoma for EDN/RB. Negative controls were obtained by omitting the primary antibody.

Immunohistochemical Quantification. Microscopic analyses were performed on a Leitz-Diaplan microscope (Nurenburg, Germany). The percentage of immunoreactive cells (nuclear staining for p60/CAF-1 and HJURP and cytoplasmic staining for PDLI4 and EDN/RB) was recorded for each staining after counting, at high power fields (×1000), 500 tumor cells in 2 different and most immunoreactive areas. Positive and negative controls were used to confirm the adequacy of staining for each run. All tissue specimens were evaluated without any knowledge of the patients' clinical information.

Statistic Methods. Selection of Cut-off Scores. The selection of clinically important cut-off scores for each protein expression was based on time-dependent ROC curve analysis. Time-dependent ROC curve analysis was performed with R software and with the survival ROC package. The prognostic accuracies of all markers were evaluated by plotting the cumulative AUC over time curve (Table 4). From the curve, the time point with the greatest accuracy for predicting survival was then identified and the 95% confidence interval (CI) for the AUC at that time point were obtained by 500-bootstraped replications of the data. The ROC curve for the marker at the time of greatest accuracy was plotted and used to identify the optimal immunohistochemical cut-off score. The optimal cut-off score was selected by identifying the point on the curve with the shortest distance to the point (0,1), or the upper-left hand corner of the ROC curve plot.

TABLE 4
Prognostic accuracy of the four markers
by time-dependent ROC curves analyses

| | Peak | | | | | |
|---|---|---|---|---|---|---|
| | accuracy | | Cut-off | Sensi- | Spec- | |
| Marker | (Months) | AUC (95%) | (%) | tivity | ificity | PPV(*) |
| EDN/RB | 39 to 55 | 0.68 | 80 | 0.59 | 0.77 | 0.72 |
| | | (0.57-0.78) | | | | |
| p60/ | 21 to 27 | 0.69 | 24 | 0.69 | 0.69 | 0.69 |
| CAF-1 | | (0.58-0.79) | | | | |
| HJURP | 28 to 29 | 0.69 | 6 | 0.92 | 0.46 | 0.63 |
| | | (0.59-0.79) | | | | |
| PDLI4 | 39 to 55 | 0.65 | 20 | 0.86 | 0.44 | 0.61 |
| | | (0.53-0.78) | | | | |

(*)PPV: Positive Predictive Value

Survival Analysis. Univariate analyses were first performed to estimate the influence of the clinical parameters and the variables EDN/RB, HJURP, p60/CAF-1 and PDLI4. Kaplan-Meier survival curves for both low and high level protein expression were analyzed by the log-rank test following the selected cut-off. Cox analysis was used to determine significance levels for each protein in a multivariate model including patient age and treatment to find a combination of independent prognostic factors. Survival analyses were carried out with R package survival.

### Results

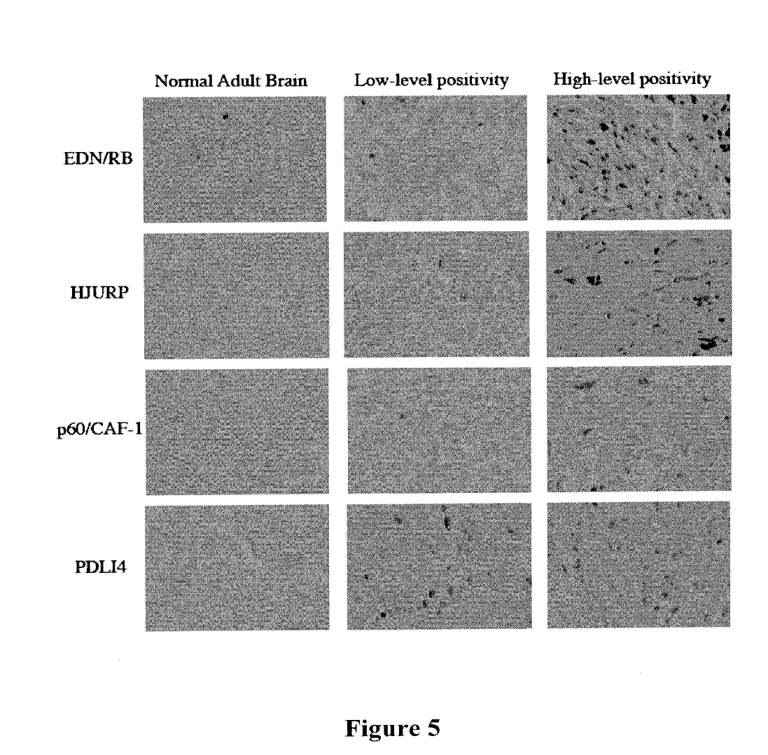

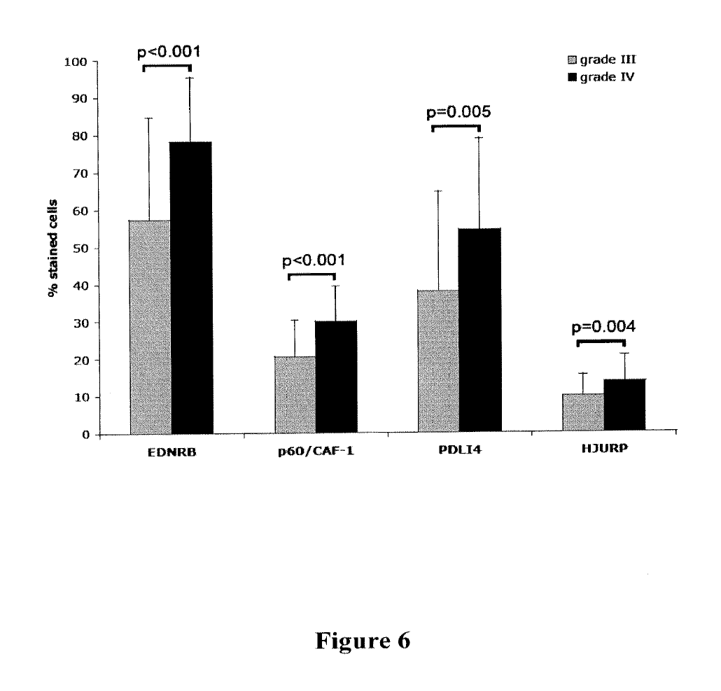

Expression of EDN/RB, p60/CAF-1, PDLI4 and HJURP distinguishes Anaplastic Gliomas from Glioblastomas. The expression of EDN/RB, p60/CAF-1, PDLI4 and HJURP proteins was evaluated by immunohistochemistry in 6 non-tumoral brain samples and 96 high-grade gliomas, including 64 glioblastomas (grade IV), 24 anaplastic astrocytomas including 10 oligoastrocytomas (grade III) and 8 anaplastic oligodendrogliomas (grade III). As shown in FIG. 5, these proteins were more expressed in high-grade gliomas compared with that in the non-tumoral brain tissue. For each protein, the expression level was significantly higher in glioblastomas compared with grade III gliomas (FIG. 6). These observations support the notion that the progression of high-grade gliomas is associated with increased EDN/RB, p60/CAF-1, PDLI4 and HJURP expression.

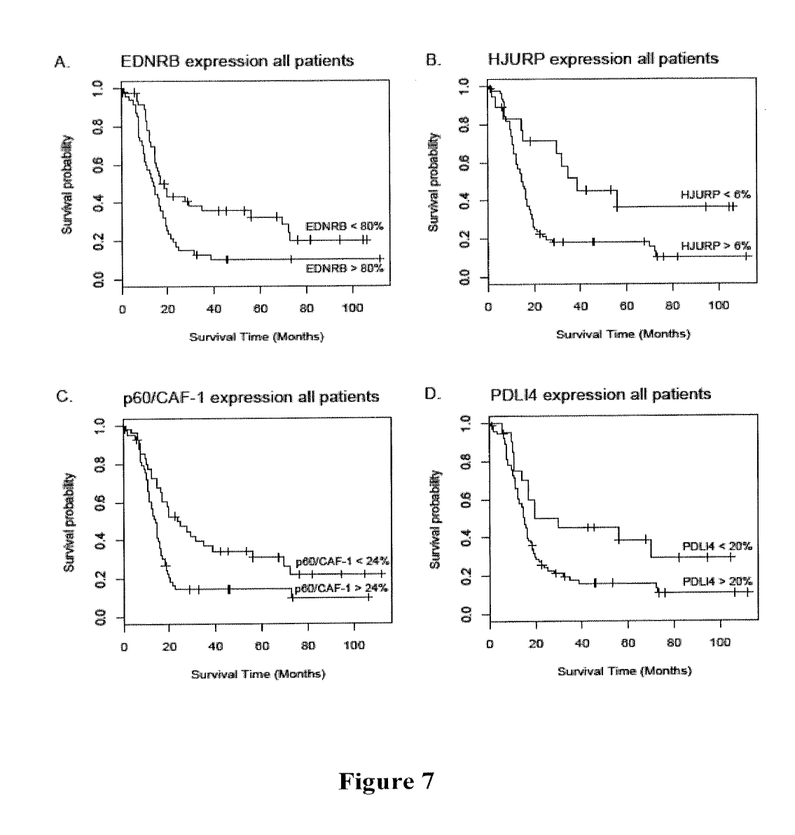

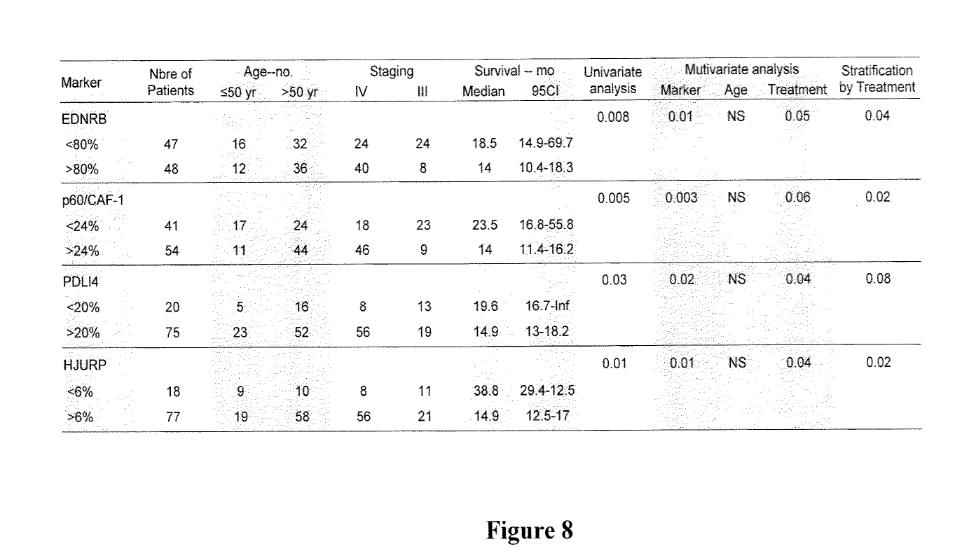

Expression of EDN/RB, p60/CAF-1, PDLI4 and HJURP is associated with Patient Prognosis. Univariate survival analysis presented in Table 3 revealed the strong associations between the overall survival and EDN/RB, p60/CAF-1 and HJURP levels, but also, in a lesser extent, that of PDLI4. For each protein, patients were stratified into two groups (high expression and low expression) according to the cut-offs defined by the examination of time-dependent ROC-curves. These cut-offs and associated performance values are summarized in Table 4. For each protein, log-rank test and Kaplan-Meier analyses showed that the stratified groups of patients had significant differences in overall survival (OS): FIG. 7 and FIG. 8. Regarding the EDN/RB protein, the median survival time of high expression level patients was 14 months (95% CI, 10.4-18.3) whereas this median for low expression level patients was 18.5 months (95% CI, 14.9-69.7). For the p60/CAF-1 protein, the difference in OS between high expression level patients and low expression level patients was also significant (14 months [95% CI, 11.4-16.2] versus 23.5 months [95% CI, 16.8-55.8]). For the PDLI4 protein, this difference was 14.9 months (95% CI, 13-18.2) versus 19.6 months (95% CI, 16.7-Inf) and still significant. The stratification following the HJURP protein level identified a long-term survivors group (38.8 months [95% CI, 29.4-12.5]). Multivariate survival analyses indicated that each of the four proteins expression levels was an independent prognostic factor for the assessment of patient outcome, and this even after adjustment for treatment (FIG. 8).

High Predictive Power of the Cumulative Study of EDN/RB, p60/CAF-1, PDLI4 and HJURP Expression. Based on these results, EDN/RB, p60/CAF-1 and HJURP were selected as the most relevant markers for HGG prognostication. A risk criterion was defined as the high level expression of at least two of these three markers. The prognostic value of this risk criterion was further evaluated. The resulting stratification provided 62 patients with a high-risk criterion and 33 patients with a low-risk criterion. These groups had a significant difference in overall survival (p<0.001) with median survival times of 14 months (95% CI, 11.4-16.2) for the high-risk group and 34.8 months (95% CI, 19.5-Inf) for the low-risk group. After adjustment for treatment, multivariate analysis confirmed that this criterion was an independent negative prognostic marker (hazard ratio=2.703; 95% CI, 1.570 to 4.653, p<0.001).

### Discussion

This study represents an extension of the study presented in Example 1. In this complementary study, the protein expression levels of the four genes that were defined as a prognostic risk panel by a meta-analysis of microarray data were analyzed. The protein expression levels were analyzed by immunohistochemistry on paraffin embedded tumor tissues. The results obtained showed that the mean expression of the EDN/RB, p60/CAF-1, PDLI4 and HJURP proteins was significantly higher in grade IV gliomas than in grade III gliomas. Up-regulation of these proteins was consistently associated with a pejorative evolution of HGGs. The combination of the EDN/RB, p60/CAF-1 and HJURP immunohistochemical results was also demonstrated to constitute an important and independent source of prognosis information for patients with HGGs. The results obtained in the genomic study showed a similar trend for CHAF1B, PDLIM4 and p60/CAF-1 in mRNA expression level but an invert correlation for EDNRB: the over-expression of EDNRB being correlated with better prognosis.

The establishment of gene classifiers in neoplastic processes and their correlation to survival or their interest in the therapeutic management of the disease is becoming increasingly common in the scientific literature in recent years (Oberthuer et al., J. Clin. Oncol., 2010, 28(21): 3506-15; Naoi et al., Breast Cancer Res. Treat. 2010 Aug. 29). In contrast, the establishment of protein classifier is much less developed with few published studies in the literature (Allory et al., Histopathology, 2008, 52(2): 158-66; Wiseman et al., Arch. Surg., 2007, 142(8): 717-27, discussion 727-9; Ring et al., Modern Pathology, 2009, 22: 1032-1043). To the best of the Applicants' knowledge, the present work provides one of the first classifiers, correlating genes and protein expression with survival in a large cohort of patients suffering from high grade gliomas.

Mismatch between protein and mRNA levels have been studied in several human tumoral processes and a variable degree of concordance is reported in the medical literature. Many of the studies suggest that external factors as well as actual biological differences between mRNA and protein abundance might affect the relationships between the two data types. Biological reasons for poor correlations include post-transcriptional and post-translational modifications, as well as the possibility that proteins have very different half-lives. Gene expression analysis is much more sensitive than immunohistochemistry but it may also be that genes are expressed at levels not high enough for translated protein expression.

The present results suggest that the progression of human HGGs is associated with up-regulation of EDN/RB, p60/CAF-1, PDLI4 and HJURP protein expression and that the expression of these proteins is tightly linked to the outcome of patients. Expression of these proteins in tumoral conditions compared to normal brain reveals a high degree of control for p60, which was not expressed in normal mature cerebral parenchyma. This particular profile is similar to the Mib1 profile with which p60 reflects the proliferative activity of the tissue sample and thus demonstrates the interest of p60 in the cerebral tumoral pathology for which any detection of p60 expression even at low levels implies a proliferative process. Under normal conditions, PDLIM4, HJURP and EDNRB are expressed and located on the cytoplasm of endothelial cells, which serves as an internal control for immunohistochemistry studies. These proteins are not expressed in the cytoplasm of astrocytes or oligodendrocytes, which demonstrates their interest in the tumoral pathology.

Very few studies of the expression of these proteins in gliomas exist in the literature. Naidoo et al. were the first to describe the overexpression of Endothelin B receptor in an inconspicuous series of low grade astrocytomas (Cancer, 2005, 104: 1049-1057). Anguelnova et al. highlighted the overexpression of Endothelin B receptor in a series of low and high grade gliomas (oligodendrogliomas, oligoastrocytomas and glioblastomas) under similar conditions to those used in the present study (immunohistochemistry on paraffin-embedded tissue) with a positivity of capillaries endothelial cells of normal brain parenchyma as external control. The distribution of positive cells and the intensity of immunostaining, however, were highly variable, both in the infiltrated tissue and the solid tumor tissue. Tumor cells exhibited variable nucleus and/or cytoplasmic labeling (Anguelova et al., Molecular Brain Research, 2005, 137: 77-88). Expression of EDN/RB has also been described in other malignant process such as malignant melanomas (Demunter et al., Virchows Arch., 2001, 438: 485-4910), bladder carcinoma (Wiilfing et al., Clin. Cancer Res., 2003, 9: 4125-31), ovarian carcinoma (Bagnato et al., Cancer Res., 1999, 59: 720-7), breast carcinoma (Wiilfing et al., European Urology, 2005, (47): 593-600) or lung carcinoma (Ahmed et al., Am. J. Respir. Cell. Mol. Biol., 2000, 22: 422-31). In malignant melanomas (MM) expression of EDN/RB rises with increasing level of invasion. Immunohistochemistry showed that primary malignant melanomas exhibited a more intense EDN/RB immunoreactivity than dysplastic nevi, whereas metastatic melanomas in turn showed a remarkably increased staining intensity relative to primary malignant melanomas. These data suggest that EDN/RB is involved in the tumor progression of malignant melanomas (Demunter et al., Virchows Arch., 2001, 438: 485-4910).

Recently, CAF-1/p60 has been proposed as a new proliferation and prognostic marker, since it has been found to be over-expressed in a series of human malignancies, in close association with their biological aggressiveness. Mascalo et al. showed an overexpression gradient of p60 between benign naevi and malignant melanomas and a significant intensity expression between radial (intraepithelial) growth and vertical (invasive) growth in malignant melanomas suggesting the prognostic accuracy of p60 expression in neoplastic process (Mascolo et al., BMC Cancer, 2010, 10: 63). CAF-1/p60 expression has also been proposed as a new tool to define the behavior of tongue (Staibano et al., Histopathology, 2007, 50: 911-919), prostatic (Staibano et al., Histopathology, 2009, 54: 580-589) or breast (Polo et al., Cancer Res., 2004, 64: 2371-2381) carcinomas.

The expression of the proteins PDLI4 and HJURP are not detailed in the literature. Nevertheless, like EDN/RB and p60/CAF-1, the concordance of the expression of PDLI4 and HJURP at the genome, transcriptome and proteome levels as well as their constant correlation with the survival of patients at these various levels demonstrates their interest in this type of pathology and the relevance of a protein scoring. Furthermore, the present protein scoring offers the advantage of being feasible on tumoral samples embedded in paraffin and does not require the use of frozen tissue which still represents one of the limits of the study of these tumors in current practice.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method for grading aggressiveness of high-grade glioma (HGG) in an individual and/or providing a HGG survival outcome to an individual, the method comprising steps of:
determining, in a biological sample obtained from the individual, expression levels of the four genes, CHAF1B, PDLIM4, EDNRB and HJURP, to obtain an expression pattern for the sample; and
based on the expression pattern obtained, grading the aggressiveness of HGG in the individual and/or providing a HGG survival outcome for the individual.

2. The method according to claim 1, wherein the individual is receiving or has received a treatment for HGG and the method is used for monitoring or assessing the effects of the treatment on HGG aggressiveness and/or HGG survival outcome in the individual treated.

3. The method according to claim 1, wherein determining the expression levels of the four genes comprises determining mRNA expression level for each of said four genes; and normalizing the mRNA expression levels determined in relation to the mRNA expression levels of one or more reference genes.

4. The method according to claim 3, wherein the reference genes are house keeping genes selected from the group consisting of B2M (beta-2 microglobulin), and HPRT1 (hypoxanthine phosphoribosyltransferase).

5. The method according to claim 3, wherein determining the expression levels of the four genes comprises performing a quantitative polymerase chain reaction or a microarray analysis.

6. The method according to claim 3, wherein overexpression of EDNRB correlates with less aggressive HGG and longer survival outcome and overexpression of CHAF1B, PDLIM4, and HJURP correlates with more aggressive HGG and shorter survival outcome.

7. The method according to claim 3, wherein determining the expression levels of the four genes further comprises calculating a gene expression risk score according to a Cox proportional hazard risk equation.

8. The method according to claim 3, further comprising a step of determining, in the biological sample, the methylation status of the MGMT promoter and/or the mutational status of IDH1.

9. The method according to claim 1, wherein determining the expression levels of the four genes comprises determining the expression levels of the four proteins, p60/CAF-1, PDLI4, EDN/RB and HJURP, encoded by the four genes.

10. The method according to claim 9, wherein determining the expression level of the four proteins comprising performing an immunoassay.

11. The method according to claim 9, wherein overexpression of the four proteins, p60/CAF-1, PDLI4, EDN/RB and HJURP, correlates with more aggressive HGG and shorter survival outcome.

12. The method of claim 1, wherein the biological sample is a fixed, paraffin-embedded tissue sample, a fresh tissue sample, or a frozen tissue sample.

13. A method for grading aggressiveness of high-grade glioma (HGG) in an individual and/or providing a HGG survival outcome to an individual, the method comprising steps of:
determining, in a biological sample obtained from the individual, expression levels:
of at least one protein selected from the group consisting of p60/CAF-1, PDLI4, EDN/RB and HJURP, or
of the three proteins: p60/CAF-1, EDN/RB and HJURP,
to obtain a protein expression pattern for the sample; and
based on the protein expression pattern obtained, grading the aggressiveness of HGG in the individual and/or providing a HGG survival outcome for the individual.

14. The method according to claim 13, wherein the individual is receiving or has received a treatment for HGG and the method is used for monitoring or assessing the effects of the treatment on HGG aggressiveness and/or HGG survival outcome in the individual treated.

15. The method according to claim 13, wherein determining the protein expression level comprises performing an immunoassay.

16. The method according to claim 13, wherein overexpression of any one of the four proteins, p60/CAF-1, PDLI4, EDN/RB and HJURP, correlates with more aggressive HGG and shorter survival outcome.

17. The method according to claim 13, wherein overexpression of the three proteins p60/CAF-1, PDLI4, EDN/RB and HJURP, correlates with more aggressive HGG and shorter survival outcome.

18. The method of claim 13, wherein the biological sample is a fixed, paraffin-embedded tissue sample, a fresh tissue sample, or a frozen tissue sample.

19. A kit for grading aggressiveness of high-grade glioma (HGG) and/or providing a HGG survival outcome to an individual, said kit comprising:
reagents that specifically detect expression levels of the four genes, CHAF1B, PDLIM4, EDNRB and HJURP, or
at least one reagent that specifically detects the expression level of at least one of the four proteins: p60/CAF-1, PDLI4, EDN/RB and HJURP; or
reagents that specifically detect expression levels of the three proteins: p60/CAF-1, EDN/RB and HJURP.

20. The kit according to claim 19 further comprising instructions for grading the aggressiveness of HGG and/or providing a HGG survival outcome to an individual according to claim 1.

21. The kit according to claim 19 further comprising instructions for grading the aggressiveness of HGG and/or providing a HGG survival outcome to an individual according to claim 13.

22. The kit according claim 19, wherein reagents that specifically detect expression levels of the four genes, CHAF1B, PDLIM4, EDNRB and HJURP, are nucleic acid probes complementary to mRNA of said genes.

23. The kit according to claim 22, wherein the nucleic acid probes complementary to mRNA of said genes are immobilized on a substrate surface.

24. The kit according claim 19, wherein the at least one reagent that specifically detects the expression level of at least one of the four proteins: p60/CAF-1, PDLI4, EDN/RB and HJURP; and the reagents that specifically detect expression levels of the three proteins: p60/CAF-1, EDN/RB and HJURP, are antibodies that specifically bind to one of the proteins.
